(19) United States
(12) Patent Application Publication
(10) Pub. No.: US 2021/0170027 A1
(43) Pub. Date: Jun. 10, 2021

(54) BIOPHOTONIC COMPOSITIONS, METHODS AND KITS FOR ENHANCING HAIR GROWTH

(71) Applicants: Giacomo ROSSI, Matelica (IT); Remigio PIERGALLINI, Grottammare (IT); Nikolaos LOUPIS, Athens (GR); Angela PALUMBO PICCIONELLO, Matelica (IT); Francesco BELLINI, Calgary (CA); Silvia SCARPONA, Matelica (IT); KLOX TECHNOLOGIES LIMITED, Dublin (IE)

(72) Inventors: Giacomo ROSSI, Matelica (IT); Remigio PIERGALLINI, Grottammare (IT); Nikolaos LOUPIS, Athens (GR); Angela PALUMBO PICCIONELLO, Matelica (IT); Francesco BELLINI, Calgary (CA); Silvia SCARPONA, Matelica (IT)

(21) Appl. No.: 16/764,673

(22) PCT Filed: Nov. 16, 2018
(86) PCT No.: PCT/CA2018/051463

§ 371 Date: May 15, 2020

Related U.S. Application Data
(60) Provisional application No. 62/587,802, filed on Nov. 17, 2017.

(51) Int. Cl. A61K 41/00; A61K 31/352; A61K 31/519; A61P 17/14

(52) U.S. Cl. CPC A61K 41/0057; A61P 17/14; A61K 31/519; A61K 31/352

(57) ABSTRACT

The technology defined in the present document generally relates to a method for enhancing hair growth on an area of a skin of a subject. The method comprises topically applying a composition comprising at least one light-absorbing molecule to the skin; and exposing said applied composition to actinic light to cause activation of the composition. Activation of the applied composition triggers enhancement of hair growth on the skin.

### CROSS-REFERENCE TO RELATED APPLICATION

This application claims the benefit of and priority to U.S. provisional patent application No. 62/587,802, filed on Nov. 17, 2017; the content of which is herein incorporated in entirety by reference.

### FIELD OF TECHNOLOGY

The present disclosure generally relates to compositions, in particular to biophotonic compositions for enhancing hair growth. The present disclosure also generally relates to methods and kits for enhancing hair growth.

### BACKGROUND INFORMATION

As follicles produce new hair cells, old cells are being pushed out through the surface of the skin at the rate of about six inches a year. Each follicle has its own life cycle that can be influenced by age, disease, and a wide variety of other factors. This life cycle of hair is divided into three phases: anagen (active hair growth that lasts between about two to six years); catagen (transitional hair growth that lasts about two to three weeks); and telogen (resting phase that lasts about two to three months; at the end of the resting phase the hair is shed and a new hair replaces it and the growing cycle starts again).

Most omnivores and herbivores (e.g., horses, cattle, pigs, rats, and mice) have simple follicles, in which each infundibulum contains 1 hair shaft (FIG. 1). Carnivores (e.g., dogs and cats) and also rabbits have compound follicles, in which each infundibulum contains multiple hair shafts. Some hair shafts in the compound follicle have a large diameter (primary or guard hairs), whereas the majority of the hair shafts have a small diameter (secondary or undercoat hairs). Humans and sheep have, depending on the body location, simple or compound follicles. In the human scalp, most follicular units are composed of 2 to 4 hairs, whereas on the body, simple hair follicles are present.

Extending vertically from the epidermis to the base of the hair follicle, the anagen hair follicle can be divided into 3 major anatomic regions (FIG. 1): (1) The infundibulum, which extends from the opening of the hair follicle to the point at which trichilemmal cornification begins (approximately the opening of the sebaceous gland duct). (2) The isthmus, which extends from the distal end of the infundibulum to the interface between the completely cornified inner root sheath and the first noncornified cell of Huxley's layer (Adamson's fringe; approximately the insertion of the arrector pili muscle). (3) The inferior portion is composed of a suprabulbar and a bulbar region and extends from the last cell in which red trichohyalin granules can be seen to the base of the hair follicle. The suprabulbar region is characterized by the noncornified inner root sheath surrounded by the outer root sheath. The hair follicle is surrounded by a basement membrane and a connective tissue sheath containing dermal sheath cells. Some of these dermal sheath cells are capable of regenerating the dermal papilla. Hair growth is needed to renew the protective covering of the body. However, hair loss is a common problem experienced by many humans as well as many animals There are many types of hair loss, also called alopecia. Involutional alopecia is a natural condition in which the hair gradually thins with age. More hair follicles go into the resting phase, and the remaining hair become shorter and fewer in numbers. Androgenic alopecia is a genetic condition that can affect both men and women. Men with this condition, called male pattern baldness, can begin suffering hair loss as early as their teens or early 20s. Androgenic alopecia is characterized by a receding hairline and gradual disappearance of hair from the crown and frontal scalp. Alopecia areata often starts suddenly and causes patchy hair loss in children and young adults. This condition may result in complete baldness (alopecia totalis). Alopecia universalis causes all body hair to fall out, including the eyebrows, eyelashes, and pubic hair. Trichotillomania, seen most frequently in children, is a psychological disorder in which a person pulls out one's own hair. Telogen effluvium is temporary hair thinning over the scalp that occurs because of changes in the growth cycle of hair. A large number of hair enters the resting phase at the same time, causing hair shedding and subsequent thinning. Scarring alopecias result in permanent loss of hair. Inflammatory skin conditions (e.g., cellulitis, folliculitis, acne), and other skin disorders (such as some forms of lupus and lichen planus) often result in scars that destroy the ability of the hair to regenerate.

One known treatment for alopecia is hair transplantation. Plugs of skin containing hair are transplanted from areas of the scalp where hair is growing to bald areas with reasonable success; however, the procedure is costly, in addition to being time-consuming and quite painful.

Non-drug related approaches to the problem include such treatments as ultra-violet radiation, massage, psychiatric and exercise therapy. None of these, however, has been generally accepted as being effective. Even such things as revascularization surgery and acupuncture have shown little, if any, promise.

The most common approach to the problem of discovering a remedy for hair loss has been drug therapy. Many types of drugs ranging from vitamins to hormones have been tried and only recently has there been any indication whatsoever of even moderate success. For instance, it was felt for a long time that since an androgenic hormone was necessary for the development of male pattern baldness, that either systemic or topical application of an antiandrogenic hormone would provide the necessary inhibiting action to keep the baldness from occurring. The theory was promising but the results were unsuccessful.

As such, there remains a need in the field of technology for compositions and methods for enhancing hair growth in a subject in need thereof that are effective, simple, easy to apply, painless, non-surgical and/or non-systemic.

### SUMMARY OF DISCLOSURE

The present technology seeks to improve at least some of the inconveniences present in the prior art.

Without wishing to be bound to any specific theory, embodiments of the present technology have been developed based on the developers' appreciation that the prior art approaches to preventing and/or treating hair loss present certain disadvantages that may benefit from being addressed.

Developers have realized that light emitted by photoactivated light-absorbing molecules could promote hair growth. Broadly speaking, embodiments of the present technology contemplate using the light-emitted by such photoactivated light-absorbing molecule to enhance hair growth on a subject.

In various aspects, the present disclosure relates to a method for enhancing hair growth on an area of a skin of a subject, comprising: topically applying a composition comprising at least one light-absorbing molecule to the skin area; and exposing said applied composition to actinic light to cause activation of the composition, wherein activation of the applied composition triggers enhancement of hair growth.

In various aspects, the present disclosure relates to the use of a composition for enhancement of hair growth in a subject in need thereof, wherein the composition comprises at least one light-absorbing molecule and a pharmaceutically acceptable carrier; and wherein the composition is suitable for topical application and for illumination by actinic light to cause activation of the composition. In various aspects, the present disclosure relates to a method for improving quality of hair on an area of skin of a subject, comprising: topically applying a composition comprising at least one light-absorbing molecule to the skin; and exposing said applied composition to actinic light to cause activation of the composition, wherein activation of the applied composition triggers improvement of the quality of hair.

In various aspects, the present disclosure relates to the use of a composition for improving quality of hair of an area of a skin of a subject, wherein the composition comprises at least one light-absorbing molecule and a pharmaceutically acceptable carrier; and wherein the composition is suitable for topical application and for illumination by actinic light to cause activation of the composition.

In various aspects, the present disclosure relates to a method for preventing and/or treating hair loss from an area of skin of a subject, comprising: topically applying a composition comprising at least one light-absorbing molecule to the skin; and exposing said applied composition to actinic light to cause activation of the composition, wherein activation of the applied composition prevents and/or treats hair loss.

In various aspects, the present disclosure relates to the use of a composition for preventing and/or treating hair loss from an area of a skin of a subject, wherein the composition comprises at least one light-absorbing molecule and a pharmaceutically acceptable carrier; and wherein the composition is suitable for topical application and for illumination by actinic light to cause activation of the composition.

In various aspects, the present disclosure relates to a method for inducing hair follicles of a skin area into anagen, comprising: topically applying a composition comprising at least one light-absorbing molecule to the skin area; and exposing said applied composition to actinic light to cause activation of the composition, wherein activation of the applied composition induces hair follicles of the skin area into anagen.

In various aspects, the present disclosure relates to the use of a composition for induction of hair follicles of a skin area into anagen, wherein the composition comprises at least one light-absorbing molecule and a pharmaceutically acceptable carrier; and wherein the composition is suitable for topical application and for illumination by actinic light to cause activation of the composition.

In various aspects, the present disclosure relates to the use of a composition for induction of hair follicles of a skin area into anagen, wherein the composition comprises at least one light-absorbing molecule; and wherein the composition is suitable for topical application and for illumination by actinic light to cause activation of the composition.

In various aspects, the present disclosure relates to the use of a composition for preventing and/or treating hair loss from an area of a skin of a subject, wherein the composition comprises at least one light-absorbing molecule; and wherein the composition is suitable for topical application and for illumination by actinic light to cause activation of the composition.

Other aspects and features of the present disclosure will become apparent to those ordinarily skilled in the art upon review of the following description of specific embodiments in conjunction with the accompanying drawings.

### BRIEF DESCRIPTION OF THE DRAWINGS

All features of embodiments which are described in this disclosure are not mutually exclusive and can be combined with one another. For example, elements of one embodiment can be utilized in the other embodiments without further mention. A detailed description of specific embodiments is provided herein below with reference to the accompanying drawings in which:

FIG. 1 depicts a schematic representation of an anagen hair follicle illustrating major anatomic regions, namely: the infundibulum, the isthmus, and the inferior portion with its suprabular and bulbar regions. The sebaceous glands and the arrector pili muscle are also illustrated.

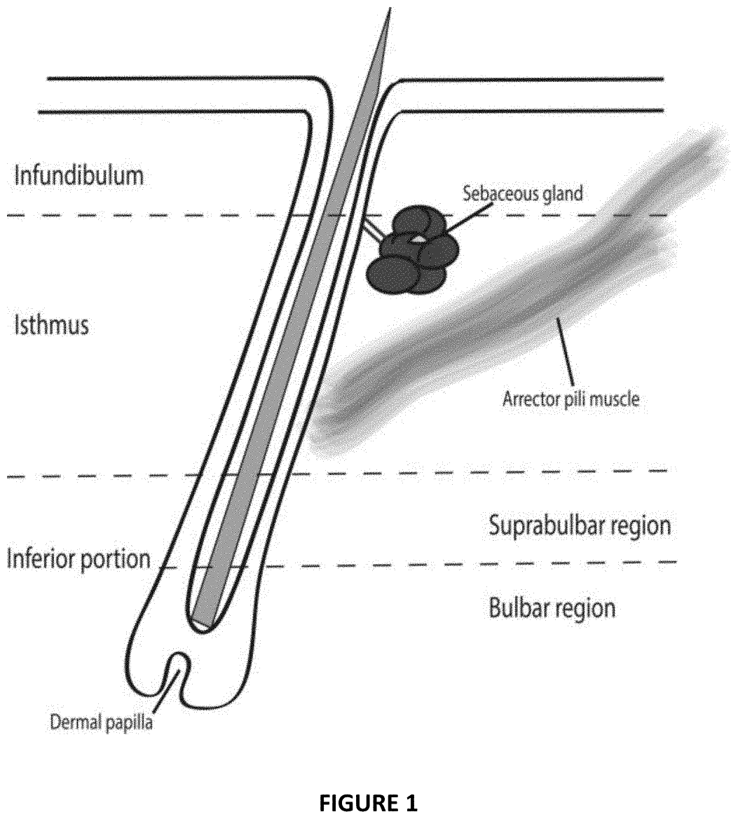

FIG. 2 depicts histological views of a skin sample after re-epithelization that has been treated with a biophotonic composition according to one embodiment of the present technology. Panel A: without biophotonic treatment; Panel B: with biophotonic treatment.

FIGS. 3A-3B depict graphs indicating the effects of treatment of a skin sample with a biophotonic composition according to one embodiment of the present technology on the number of anagenic follicule (FIG. 3A) and on VEGF expression (FIG. 3B). Control: without biophotonic treatment; Treated: with biophotonic treatment.

FIG. 4 depicts histological views of a high power magnification of follicular area dermis of a skin sample after re-epithelization. The histological views show the effect of treatment of the skin sample with the biophotonic composition according to one embodiment of the present technology on EGF expression. Panel A: without biophotonic treatment; Panel B: with biophotonic treatment.

FIG. 5 depicts histological views of a skin sample after re-epithelization that has been treated with a biophotonic composition according to one embodiment of the present technology. Effect of the treatment on EGF expression (Panel A); VEGF expression (Panel B); Ki67 (Mib/1) expression; and on the number of follicles with vascula pailla (Panel D).

FIG. 6 depicts graphs showing the effect of treatment of a skin sample obtained from a first subject with a biophotonic composition according to one embodiment of the present technology on histology (Panel A); FVIII expression (Panel B); TNFalpha expression (Panel C); FGF expression (Panel D); TGFbeta expression (Panel E); EGF expression (Panel F); decorin expression (Panel G); Ki67 expression (Panel H); collagen III expression (Panel I); and Hsp60 expression (Panel J). Control: without biophotonic treatment; Treated: with biophotonic treatment.

FIG. 7 depicts graphs showing the effect of treatment of a skin sample obtained from a second subject with a biophotonic composition according to one embodiment of the present technology on histology (Panel A); FVIII expression (Panel B); TNFalpha expression (Panel C); FGF expression (Panel D); TGFbeta expression (Panel E); EGF expression (Panel F); decorin expression (Panel G); Ki67 expression (Panel H); collagen III expression (Panel I); and Hsp60 expression (Panel J). Control: without biophotonic treatment; Treated: with biophotonic treatment.

FIG. 8 depicts graphs showing the effect of treatment of a skin sample obtained from a third subject with a biophotonic composition according to one embodiment of the present technology on histology (Panel A); FVIII expression (Panel B); TNFalpha expression (Panel C); FGF expression (Panel D); TGFbeta expression (Panel E); EGF expression (Panel F); decorin expression (Panel G); Ki67 expression (Panel H); collagen III expression (Panel I); and Hsp60 expression (Panel J). Control: without biophotonic treatment; Treated: with biophotonic treatment.

FIG. 9 depicts graphs showing the effect of treatment of a skin sample obtained from a fourth subject with a biophotonic composition according to one embodiment of the present technology on histology (Panel A); FVIII expression (Panel B); TNFalpha expression (Panel C); FGF expression (Panel D); TGFbeta expression (Panel E); EGF expression (Panel F); decorin expression (Panel G); Ki67 expression (Panel H); collagen III expression (Panel I); and Hsp60 expression (Panel J). Control: without biophotonic treatment; Treated: with biophotonic treatment.

FIG. 10 depicts graphs showing the effect of treatment of a skin sample obtained from a fifth subject with a biophotonic composition according to one embodiment of the present technology on histology (Panel A); FVIII expression (Panel B); TNFalpha expression (Panel C); FGF expression (Panel D); TGFbeta expression (Panel E); EGF expression (Panel F); decorin expression (Panel G); Ki67 expression (Panel H); collagen III expression (Panel I); and Hsp60 expression (Panel J). Control: without biophotonic treatment; Treated: with biophotonic treatment.

### DETAILED DISCLOSURE OF EMBODIMENTS

The present technology is explained in greater detail below. This description is not intended to be a detailed catalog of all the different ways in which the technology may be implemented, or all the features that may be added to the instant technology. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which variations and additions do not depart from the present technology. Hence, the following description is intended to illustrate some particular embodiments of the technology, and not to exhaustively specify all permutations, combinations and variations thereof.

As used herein, the singular form “a,” “an” and “the” include plural referents unless the context clearly dictates otherwise.

The recitation herein of numerical ranges by endpoints is intended to include all numbers subsumed within that range (e.g., a recitation of 1 to 5 includes 1, 1.5, 2, 2.75, 3, 3.80, 4, 4.32, and 5).

The term “about” is used herein explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. For example, the term “about” in the context of a given value or range refers to a value or range that is within 20%, preferably within 15%, more preferably within 10%, more preferably within 9%, more preferably within 8%, more preferably within 7%, more preferably within 6%, and more preferably within 5% of the given value or range.

The expression “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

As used herein, the term “hair” refers to the protein filament that grows from follicles found in the dermis and includes scalp, head, facial, and/or body hair, eye lashes, brows, mustache, beard, ear, nasal, chest, pubic, auxiliary, fur, and the like.

The terms “grow” or “growth” as used herein with respect to hair means the growth or regrowth of hair. Accordingly, the terms “growth” and “regrowth” are used interchangeably with respect to growing hair.

By “enhancing hair growth” or “stimulating hair growth” or “inducing hair growth” or “promoting hair growth” is meant the earlier induction of growth of a new hair cycle, and/or prolonging the active growth phase (anagen) of the hair cycle, and/or increasing the growth rate of the hair, and/or increasing the width of the hair shaft, including, but not limited to, the induction of the growth of hair and making it more visible to the eye.

As used herein “improving hair quality” means increasing the diameter of the hair shaft and/or enhancing the visual attributes of the hair like hair volume, hair shine, and hair thickness, and/or affecting the characteristics of the hair shaft, and/or hair cuticles, including, but not limited to, creating a smoother look or feel, and/or increase in shine.

As used herein, the expression “visual inspection” means that a human viewer can visually discern the presence of hair or hair growth with the unaided eye (excepting standard corrective lenses adapted to compensate for near-sightedness, farsightedness, or stigmatism, or other corrected vision) in lighting at least equal to the illumination of a standard 75 watt incandescent white light bulb at a distance of about 0.25 meter.

The term “biophotonic” as used herein refers to the generation, manipulation, detection and application of photons in a biologically relevant context. As used herein, the expression “biophotonic composition” refers to a composition as described herein that may be activated by light to produce photons for biologically relevant applications.

The term “topical” means as applied to body surfaces, such as the skin, mucous membranes, vagina, oral cavity, internal surgical wound sites, and the like.

Terms and expressions “light-absorbing molecule”, “light-capturing molecule”, “photoactivating agent”, “chromophore” and “photoactivator” are used herein interchangeably. A light-absorbing molecule means a molecule or a complex of molecules, which when contacted by light irradiation, is capable of absorbing the light. The light-absorbing molecule readily undergoes photoexcitation and in some instances transfers its energy to other molecules or emits it as light.

The term “gels” as used herein refers to substantially dilute cross-linked systems. Gels may be semi-solids and exhibit substantially no flow when in the steady state at room temperature (e.g. about 20-25° C.). By steady state is meant herein during a treatment time and under treatment conditions. Gels, as defined herein, may be physically or chemically cross-linked. As defined herein, gels also include gel-like compositions such as viscous liquids.

The term “membrane” as used in the expression “biophotonic membrane” refers to a biophotonic composition which is in the form of a membrane containing at least one light-absorbing molecule. The biophotonic membranes of the present disclosure may be deformable. They may be elastic or non-elastic (i.e. flexible or rigid). The biophotonic membrane, for example, may be in a peel-off form (‘peelable’) to provide ease and speed of use. In certain instances, the tear strength and/or tensile strength of the peel-off form is greater than its adhesion strength. This may help handleability of the biophotonic membrane. In some instances, the biophotonic membrane comprises silicone. In some instances, the biophotonic membrane comprises a thermogelling solution.

The expression “healing factor” as used herein refers to a compound that promotes or enhances the healing or regenerative process of a tissue.

The “initial level of fluorescence” is the level of fluorescence exhibited by a composition of the present disclosure immediately upon application of or activation with light.

The term “photobleaching” as used herein refers to the photochemical destruction of a light-absorbing molecule.

The expression “actinic light” as used herein refers to light energy emitted from a specific light source (e.g., lamp, LED, or laser) and capable of being absorbed by matter (e.g., the light-absorbing molecule defined above). In some embodiments, the actinic light is visible light.

As used herein, the term “treated” in expressions such as: “treated skin”, “treated tissue”, and “treated area/portion of the skin”, refers to a skin or tissue onto which a method according to the embodiments of the present disclosure has been performed. For example, in some instances, a treated skin refers to a skin onto which the composition of the present disclosure has been applied and which has been illuminated as outlined herein.

In one embodiment, the present disclosure relates to a method for enhancing hair growth in a subject. The method comprises applying a composition comprising at least one light-absorbing molecule to the subject and exposing the applied composition to actinic light to cause activation of the composition.

In one embodiment, the present disclosure relates to a method for enhancing hair growth in a subject. The method comprises applying a biophotonic composition comprising at least one light-absorbing molecule to the subject and exposing the applied biophotonic composition to actinic light to cause activation of the biophotonic composition.

In one embodiment, the present disclosure relates to a method for enhancing hair growth of a skin area on a subject in need of hair growth enhancement. The method comprises applying a biophotonic composition comprising at least one light-absorbing molecule to the skin area of the subject in need thereof and exposing the applied biophotonic composition to actinic light to cause activation of the biophotonic composition.

In one embodiment, the present disclosure relates to a method for enhancing hair growth of a skin area on a subject in need of hair growth enhancement, wherein the skin area comprises wounded and/or scarred tissue. The method comprises applying a biophotonic composition comprising at least one light-absorbing molecule to the skin area of the subject in need thereof and exposing the applied biophotonic composition to actinic light to cause activation of the biophotonic composition.

In one embodiment, the present disclosure relates to a method for improving hair quality on a subject. The method comprises applying a composition comprising at least one light-absorbing molecule to the subject and exposing the applied composition to actinic light to cause activation of the composition.

In one embodiment, the present disclosure relates to a method for improving hair quality on a subject. The method comprises applying a biophotonic composition comprising at least one light-absorbing molecule to the subject and exposing the applied biophotonic composition to actinic light to cause activation of the biophotonic composition.

In one embodiment, the present disclosure relates to a method for improving the hair quality on a subject in need of hair quality improvement. The method comprises applying a biophotonic composition comprising at least one light-absorbing molecule to the skin area of the subject in need thereof and exposing the applied biophotonic composition to actinic light to cause activation of the biophotonic composition.

In one embodiment, the present disclosure relates to a method for improving the overall quality of hair of a subject in need of hair quality improvement, wherein the skin area comprises wounded and/or scarred tissue. The method comprises applying a biophotonic composition comprising at least one light-absorbing molecule to the skin area of the subject in need thereof and exposing the applied biophotonic composition to actinic light to cause activation of the biophotonic composition.

In one embodiment, the present disclosure relates to a method for preventing and/or treating hair loss on a subject. The method comprises applying a composition comprising at least one light-absorbing molecule to the subject and exposing the applied composition to actinic light to cause activation of the composition.

In one embodiment, the present disclosure relates to a method for preventing and/or treating hair loss on a subject. The method comprises applying a biophotonic composition comprising at least one light-absorbing molecule to the subject and exposing the applied biophotonic composition to actinic light to cause activation of the biophotonic composition.

In one embodiment, the present disclosure relates to a method for preventing and/or treating hair loss on a subject in need of hair loss prevention and/or treatment. The method comprises applying a biophotonic composition comprising at least one light-absorbing molecule to the skin area of the subject in need thereof and exposing the applied biophotonic composition to actinic light to cause activation of the biophotonic composition.

In one embodiment, the present disclosure relates to a method for preventing and/or treating hair loss on a subject in need of hair loss prevention and/or treatment, wherein the skin area comprises wounded and/or scarred tissue. The method comprises applying a biophotonic composition comprising at least one light-absorbing molecule to the skin area of the subject in need thereof and exposing the applied biophotonic composition to actinic light to cause activation of the biophotonic composition.

In some implementations of these embodiments, the enhancement of hair growth is achieved non-systemically, that is to say that, in these implementations, the hair growth enhancement is achieved without using substances that travel through the bloodstream of the subject.

In some other implementations of these embodiments, the light-absorbing molecules are exogenous from the skin or tissue onto which the light-absorbing molecules are to be applied (i.e., light-absorbing molecules that are not naturally present in skin or tissue onto which the composition as defined herein is to be applied).

Compositions

In some embodiments, the present disclosure provides compositions for enhancing hair growth. In some implementations of these embodiments, the composition of the present disclosure is a biophotonic composition.

The compositions of the present disclosure are activated by light (e.g., photons) of specific wavelength. The compositions comprise at least one exogenous light-absorbing molecule which is activated by light and accelerates the dispersion of light energy, which leads to light carrying on a therapeutic effect on its own, and/or to the photochemical activation of other agents present in the composition.

When a light-activating molecule absorbs a photon of a certain wavelength, it becomes excited. This is an unstable condition and the light-activating molecule tries to return to the ground state, giving away the excess energy. For some light-activating molecules, it is favorable to emit the excess energy as light when transforming back to the ground state. This process is called fluorescence. The peak wavelength of the emitted fluorescence is shifted towards longer wavelengths compared to the absorption wavelengths (i.e., Stokes' shift). The emitted fluorescent energy can then be transferred to the other components of the composition or to a treatment site on to which the composition is topically applied. Differing wavelengths of light may have different and complementary therapeutic effects on tissue.

In certain instances, the compositions of the present disclosure are substantially transparent. In certain instances, the compositions of the present disclosure are translucent. In certain instances, the compositions of the present disclosure have high light transmittance in order to permit light dissipation into and through the composition. In this way, the area of tissue under the composition can be treated both with the fluorescent light emitted by the composition and the light irradiating the composition to activate it, which may benefit from the different therapeutic effects of light having different wavelengths.

The % transmittance of the composition can be measured in the range of wavelengths from 250 nm to 800 nm using, for example, a Perkin-Elmer Lambda 9500 series UV-visible spectrophotometer. Alternatively, a Synergy HT spectrophotometer (BioTek Instrument, Inc.) can be used in the range of wavelengths from 380 nm to 900 nm. Transmittance is calculated according to the following equation:

$${A}_{λ} = {log}_{10} \frac{{I}_{0}}{I} = {log}_{10} \frac{1}{T} ,$$

where A is absorbance, T is transmittance, I₀ is intensity of radiation before passing through material, and I is intensity of light passing through material. The values can be normalized for thickness. As stated herein, % transmittance (translucency) is as measured for a 2 mm thick sample at a wavelength of 526 nm. It will be clear that other wavelengths can be used.

In some embodiments, the composition has a transparency or translucency that exceeds about 15%, about 20%, about 25%, about 30%, about 35%, about 40%, about 45%, about 50%, about 55%, about 60%, about 65%, about 70%, about 75%, about 80%, or about 85%. In some embodiments, the transparency exceeds about 70%, about 80%, about 85%, about 86%, about 87%, about 88%, about 89%, about 90%, about 91%, about 92%, about 93%, about 94%, about 95%, about 96%, about 97%, about 98% or about 99%. All transmittance values reported herein are as measured on a 2 mm thick sample using the Synergy HT spectrophotometer at a wavelength of 526 nm.

In some instances, the compositions of the present disclosure are for topical uses (i.e., suitable for topical application). The composition can be in the form of a semi-solid or viscous liquid, such as a gel, or are gel-like, and which have a spreadable consistency at room temperature (e.g., about 20-25° C.) prior to illumination. In certain such instances wherein the composition has a spreadable consistency, the composition can be topically applied to a treatment site at a thickness of from about 0.5 mm to about 3 mm, or from about 0.5 mm to about 2.5 mm, or from about 1 mm to about 2 mm. The composition can be topically applied to a treatment site at a thickness of about 2 mm or about 1 mm Spreadable compositions can conform to a topography of an application site. This can have advantages over a non-conforming material in that a better and/or more complete illumination of the application site can be achieved and the compositions are easy to apply and remove.

In some aspects, the compositions of the present disclosure comprise at least a first light-absorbing molecule in a medium, wherein the composition is substantially resistant to leaching such that a low or negligible amount of the light-absorbing molecule leaches out of the composition into for example skin or onto a soft tissue onto which the composition is applied. In certain embodiments, this is achieved by the medium comprising a gelling agent which slows or restricts movement or leaching of the light-absorbing molecule.

In some aspects, the compositions of the present disclosure do not stain the tissue onto which they are topically applied. Staining is determined by visually assessing whether the composition colorizes white test paper saturated with 70% by volume ethanol/30% by volume water solution placed in contact with the composition for a period of time corresponding to a desired illumination time. In some embodiments, a composition of the present disclosure does not visually colorize white test paper saturated with a 70% by volume ethanol/30% by volume water solution placed in contact with the composition under atmospheric pressure for a time corresponding to a desired illumination time.

Suitable light-absorbing molecules can be fluorescent dyes (or stains), although other dye groups or dyes (biological and histological dyes, food colorings, carotenoids, and other dyes) can also be used. Suitable light-absorbing molecules can be those that are Generally Regarded As Safe (GRAS), although light-absorbing molecules which are not well tolerated by the skin or other tissues can be included in the composition as contact with the skin is minimal in use due to the leaching-resistant nature of the composition.

In certain embodiments, the composition of the present disclosure comprises at least one light-absorbing molecule which undergoes partial or complete photobleaching upon application of light. In some embodiments, the at least one light-absorbing molecule absorbs and/or emits at a wavelength in the range of the visible spectrum, such as at a wavelength of between about 380 nm and about 1 mm, between about 380 nm and about 800 nm, between about 380 nm and about 700 nm, or between about 380 nm and about 600 nm. In other embodiments, the at least one light-absorbing molecule absorbs/or emits at a wavelength of between about 20 nm and about 1 mm, between about 200 nm and about 800 nm, between about 200 nm and about 700 nm, between about 200 nm and about 600 nm or between about 200 nm and about 500 nm. In other embodiments, the at least one light-absorbing molecule absorbs/or emits at a wavelength of between about 200 nm and about 600 nm. In some embodiments, the at least one light-absorbing molecule absorbs/or emits light at a wavelength of between about 200 nm and about 300 nm, between about 250 nm and about 350 nm, between about 300 nm and about 400 nm, between about 350 nm and about 450 nm, between about 400 nm and about 500 nm, between about 400 nm and about 600 nm, between about 450 nm and about 650 nm, between about 600 nm and about 700 nm, between about 650 nm and about 750 nm or between about 700 nm and about 800 nm.

It will be appreciated to those skilled in the art that optical properties of a particular light-absorbing molecule may vary depending on the light-absorbing molecule's surrounding medium. Therefore, as used herein, a particular light-absorbing molecule's absorption and/or emission wavelength (or spectrum) corresponds to the wavelengths (or spectrum) measured in a composition useful in the methods of the present disclosure.

In some instances, the light-absorbing molecule of the composition is one or more of: a xanthene derivative dye, an azo dye, a biological stain, and a carotenoid. In some instances, the at least one light-absorbing molecule is selected from eosin (e.g., eosin B or eosin Y), erythrosine (e.g., erythrosine B), fluorescein, Rose Bengal, and Saffron red powder.

In certain such embodiments, said xanthene derivative dye is chosen from a fluorene dye (e.g., a pyronine dye, such as pyronine Y or pyronine B, or a rhodamine dye, such as rhodamine B, rhodamine G, or rhodamine WT), a fluorone dye (e.g., fluorescein, or fluorescein derivatives, such as phloxine B, rose bengal, merbromine, Eosin Y, Eosin B, or Erythrosine B), or a rhodole dye. In certain such embodiments, said azo dye is chosen from methyl violet, neutral red, para red, amaranth, carmoisine, allura red AC, tartrazine, orange G, ponceau 4R, methyl red, and murexide-ammonium purpurate. In certain such embodiments, said biological stain is chosen from safranin O, basic fuchsin, acid fuschin, 3,3′ dihexylocarbocyanine iodide, carminic acid, and indocyanine green. In certain such embodiments, said carotenoid is chosen from crocetin, a-crocin (S,S-diapo-S,S-carotenoic acid), zeaxanthine, lycopene, α-carotene, β-carotene, bixin, and fucoxanthine. In certain such embodiments, said carotenoid is present in the composition as a mixture is selected from saffron red powder, annatto extract, and brown algae extract.

In some embodiments, the at least one light-absorbing molecule is present in an amount of between about 0.001% and about 40% by weight of the composition. In some embodiments, the at least one light-absorbing molecule is present in an amount of between about 0.005% and about 2%, between about 0.01% and about 1%, between about 0.01% and about 2%, between about 0.05% and about 1%, between about 0.05% and about 2%, between about 0.1% and about 1%, between about 0.1% and about 2%, between about 1% and about 5%, about 2.5% and about 7.5%, between about 5% and about 10%, between about 7.5% and about 12.5%, between about 10% and about 15%, between about 12.5% and about 17.5%, between about 15% and about 20%, between about 17.5% and about 22.5%, between about 20% and about 25%, between about 22.5% and about 27.5%, between about 25% and about 30%, between about 27.5% and about 32.5%, between about 30% and about 35%, between about 32.5% and about 37.5%, or between about 35% and about 40% by weight of the composition. In some embodiments, the at least one light-absorbing molecule is present in an amount of at least about 0.2% by weight of the composition.

In some embodiments, the at least one light-absorbing molecule is present in an amount of between about 0.001% and about 40% by weight of the composition. In some embodiments, the at least one light-absorbing molecule is present in an amount of between about 0.005% and about 2%, between about 0.01% and about 1%, between about 0.01% and about 2%, between about 0.05% and about 1%, between about 0.05% and about 2%, between about 0.1% and about 1%, between about 0.1% and about 2%, between about 1% and about 5%, between about 2.5% and about 7.5%, between about 5% and about 10%, between about 7.5% and about 12.5%, between about 10% and about 15%, between about 12.5% and about 17.5%, between about 15% and about 20%, between about 17.5% and about 22.5%, between about 20% and about 25%, between about 22.5% and about 27.5%, between about 25% and about 30%, between about 27.5% and about 32.5%, between about 30% and about 35%, between about 32.5% and about 37.5%, or between about 35% and about 40% by weight of the composition. In some embodiments, the at least one light-absorbing molecule is present in an amount of at least about 0.2% by weight of the composition.

The compositions disclosed herein may include at least one additional light-absorbing molecule. Combining light-absorbing molecules may increase photo-absorption by the combined dye molecules and enhance absorption and photo-biomodulation selectivity. This creates multiple possibilities of generating new photosensitive, and/or selective light-absorbing molecule mixtures.

When such multi-light-absorbing molecule compositions are illuminated with light, energy transfer can occur between the light-absorbing molecules. This process, known as resonance energy transfer, is a photophysical process through which an excited ‘donor’ light-absorbing molecule (also referred to herein as first light-absorbing molecule) transfers its excitation energy to an ‘acceptor’ light-absorbing molecule (also referred to herein as second light-absorbing molecule). The efficiency and directedness of resonance energy transfer depends on the spectral features of donor and acceptor light-absorbing molecule. In particular, the flow of energy between light-absorbing molecules is dependent on a spectral overlap reflecting the relative positioning and shapes of the absorption and emission spectra. For energy transfer to occur the emission spectrum of the donor light-absorbing molecule overlap with the absorption spectrum of the acceptor light-absorbing molecule. Energy transfer manifests itself through decrease or quenching of the donor emission and a reduction of excited state lifetime accompanied also by an increase in acceptor emission intensity. To enhance the energy transfer efficiency, the donor chromophore should have good abilities to absorb photons and emit photons. Furthermore, it is thought that the more overlap there is between the donor light-absorbing molecule's emission spectra and the acceptor light-absorbing molecule's absorption spectra, the better a donor light-absorbing molecule can transfer energy to the acceptor light-absorbing molecule.

In some embodiments, the donor, or first, light-absorbing molecule has an emission spectrum that overlaps at least about 80%, about 70%, about 60%, about 50%, about 40%, about 30%, about 20%, or about 10% with an absorption spectrum of the second light-absorbing molecule. In some embodiments, the first light-absorbing molecule has an emission spectrum that overlaps at least about 20% with an absorption spectrum of the second light-absorbing molecule. In some embodiments, the first light-absorbing molecule has an emission spectrum that overlaps at least between about 1% and about 10%, between about 5% and about 15%, between about 10% and about 20%, between about 15% and about 25%, between about 20% and about 30%, between about 25% and about 35%, between about 30% and about 40%, between about 35% and about 45%, between about 50% and about 60%, between about 55% and about 65% or between about 60% and about 70% with an absorption spectrum of the second light-absorbing molecule.

Percent (%) spectral overlap, as used herein, refers to the % overlap of a donor light-absorbing molecule's emission wavelength range with an acceptor light-absorbing molecule's absorption wavelength range, measured at spectral full width quarter maximum (FWQM).

In some embodiments, the second light-absorbing molecule absorbs at a wavelength in the range of the visible spectrum. In some embodiments, the second light-absorbing molecule has an absorption wavelength that is relatively longer than that of the first light-absorbing molecule within the range of between about 50 nm and about 250 nm, between about 25 nm and about 150 nm or between about 10 nm and about 100 nm.

As discussed above, the application of light to the compositions of the present disclosure can result in a cascade of energy transfer between the light-absorbing molecules. In some embodiments, such a cascade of energy transfer provides photons that penetrate the epidermis, dermis and/or mucosa at the target tissue.

In some embodiments, the light-absorbing molecule is selected such that their emitted fluorescent light, on photoactivation, is within one or more of the green, yellow, orange, red and infrared portions of the electromagnetic spectrum, for example having a peak wavelength within the range of about 490 nm to about 800 nm. In some embodiments, the emitted fluorescent light has a power density of between 0.005 mW/cm² to about 10 mW/cm², about 0.5 mW/cm² to about 5 mW/cm².

Further examples of suitable light-absorbing molecules useful in the compositions, methods, and uses of the present disclosure include, but are not limited to the following: Xanthene derivatives—The xanthene group comprises three sub-groups: a) the fluorenes; b) fluorones; and c) the rhodoles, any of which may be suitable for the compositions, methods, and uses of the present disclosure. The fluorenes group comprises the pyronines (e.g., pyronine Y and B) and the rhodamines (e.g., rhodamine B, G and WT). Depending on the concentration used, both pyronines and rhodamines may be toxic and their interaction with light may lead to increased toxicity. Similar effects are known to occur for the rhodole dye group. The fluorone group comprises the fluorescein dye and the fluorescein derivatives. Fluorescein is a fluorophore commonly used in microscopy with an absorption maximum of 494 nm and an emission maximum of 521 nm. The disodium salt of fluorescein is known as D&C Yellow 8. It has very high fluorescence but photodegrades quickly. In the present composition, mixtures of fluorescein with other photoactivators such as indocyanin green and/or saffron red powder will confer increased photoabsorption to these other compounds. The eosins group comprises Eosin Y (tetrabromofluorescein, acid red 87, D&C Red 22), a chromophore with an absorption maximum of 514-518 nm that stains the cytoplasm of cells, collagen, muscle fibers and red blood cells intensely red; and Eosin B (acid red 91, eosin scarlet, dibromo-dinitrofluorescein), with the same staining characteristics as Eosin Y. Eosin Y and Eosin B are collectively referred to as “Eosin”, and use of the term “Eosin” refers to either Eosin Y, Eosin B or a mixture of both. Eosin Y, Eosin B, or a mixture of both can be used because of their sensitivity to the light spectra used: broad spectrum blue light, blue to green light and green light. In some embodiments, the composition includes in the range of less than about 12% by weight of the total composition of at least one of Eosin B or Eosin Y or a combination thereof. In some embodiments, at least one of Eosin B or Eosin Y or a combination thereof is present from about 0.001% to about 12%, or between about 0.01% and about 1.2%, or from about 0.01% to about 0.5%, or from about 0.01% to about 0.05%, or from about 0.1% to about 0.5%, or from about 0.5% to about 0.8% by weight of the total composition. In some embodiments, at least one of Eosin B or Eosin Y or a combination thereof is present is an amount of at about 0.005% by weight of the total composition. In some embodiments, at least one of Eosin B or Eosin Y or a combination thereof is present is an amount of at about 0.01% by weight of the total composition. In some embodiments, at least one of Eosin B or Eosin Y or a combination thereof is present is an amount of at about 0.02% by weight of the total composition. In some embodiments, at least one of Eosin B or Eosin Y or a combination thereof is present is an amount of at about 0.05% by weight of the total composition. In some embodiments, at least one of Eosin B or Eosin Y or a combination thereof is present is an amount of at about 0.1% by weight of the total composition. In some embodiments, at least one of Eosin B or Eosin Y or a combination thereof is present is an amount of at about 0.2% by weight of the total composition. In some embodiments, at least one of Eosin B or Eosin Y or a combination thereof is present is an amount of at least about 0.2% by weight of the total composition but less than about 1.2% by weight of the total composition. In some embodiments, at least one of Eosin B or Eosin Y or a combination thereof is present is an amount of at least about 0.01% by weight of the total composition but less than about 12% by weight of the total composition. In some embodiments, the composition includes in the range of less than about 12% by weight of the total composition of at least one of Eosin B or Eosin Y or a combination thereof. In some embodiments, at least one of Eosin B or Eosin Y or a combination thereof is present from about 0.001% to about 12%, or between about 0.01% and about 1.2%, or from about 0.01% to about 0.5%, or from about 0.1% to about 0.5%, or from about 0.5% to about 0.8%, or from about 0.01% to about 0.05%, by weight of the total composition. In some embodiments, at least one of Eosin B or Eosin Y or a combination thereof is present is an amount of at least about 0.005% by weight of the total composition. In some embodiments, at least one of Eosin B or Eosin Y or a combination thereof is present is an amount of at least about 0.01% by weight of the total composition. In some embodiments, at least one of Eosin B or Eosin Y or a combination thereof is present is an amount of at least about 0.02% by weight of the total composition. In some embodiments, at least one of Eosin B or Eosin Y or a combination thereof is present is an amount of at least about 0.05% by weight of the total composition. In some embodiments, at least one of Eosin B or Eosin Y or a combination thereof is present is an amount of at least about 0.1% by weight of the total composition. In some embodiments, at least one of Eosin B or Eosin Y or a combination thereof is present is an amount of at least about 0.2% by weight of the total composition. In some embodiments, at least one of Eosin B or Eosin Y or a combination thereof is present is an amount of at least about 0.2% by weight of the total composition but less than about 1.2% by weight of the total composition. In some embodiments, at least one of Eosin B or Eosin Y or a combination thereof is present is an amount of at least about 0.01% by weight of the total composition but less than about 12% by weight of the total composition. Phloxine B (2,4,5,7 tetrabromo 4,5,6,7,tetrachlorofluorescein, D&C Red 28, acid red 92) is a red dye derivative of fluorescein which is used for disinfection and detoxification of waste water through photooxidation. It has an absorption maximum of 535-548 nm. It is also used as an intermediate for making photosensitive dyes and drugs. Erythrosine B, or simply Erythrosine or Erythrosin (acid red 51, tetraiodofluorescein) is a cherry-pink, coal-based fluorine food dye used as a biological stain, and a biofilm and dental plaque disclosing agent, with a maximum absorbance of 524-530 nm in aqueous solution. It is subject to photodegradation. Erythrosine is also used in some embodiments due to its photosensitivity to the light spectra used and its ability to stain biofilms. In embodiments, the composition includes in the range of less than about 2% by weight Erythrosine B. In some embodiments, Erythrosine B is present in an amount from about 0.005% to about 2%, or from about 0.005% to about 1%, or about 0.01% to about 1% by weight of the total composition. In some embodiments, Erythrosine B is present in an amount of about 0.005% and about 0.15% by weight of the total composition. Rose Bengal (4,5,6,7 tetrachloro 2,4,5,7 tetraiodofluorescein, acid red 94) is a bright bluish-pink fluorescein derivative with an absorption maximum of 544-549 nm, that has been used as a dye, biological stain and diagnostic aid. Merbromine (mercurochrome) is an organo-mercuric disodium salt of fluorescein with an absorption maximum of 508 nm. It is used as an antiseptic. Suitable azo dyes for the compositions, methods, and uses of the disclosure include: Methyl violet, neutral red, para red (pigment red 1), amaranth (Azorubine S), Carmoisine (azorubine, food red 3, acid red 14), allura red AC (FD&C 40), tartrazine (FD&C Yellow 5), orange G (acid orange 10), Ponceau 4R (food red 7), methyl red (acid red 2), and murexide-ammonium purpurate. Suitable biological stains include: Safranin (Safranin 0, basic red 2) is an azo-dye and is used in histology and cytology. It is a classic counter stain in a Gram stain protocol. Fuchsin (basic or acid) (rosaniline hydrochloride) is a magenta biological dye that can stain bacteria and has been used as an antiseptic. It has an absorption maximum of 540-555 nm. 3,3′ dihexylocarbocyanine iodide (DiOC6) is a fluorescent dye used for staining the endoplasmic reticulum, vesicle membranes and mitochondria of cells. Carminic acid (acid red 4, natural red 4) is a red glucosidal hydroxyanthrapurin naturally obtained from cochineal insects. Indocyanin green (ICG) is used as a diagnostic aid for blood volume determination, cardiac output, or hepatic function. ICG binds strongly to red blood cells and when used in mixture with fluorescein, it increases the absorption of blue to green light. Carotenoids—Carotenoid dyes are also photoactivators that are useful in the compositions, methods, and uses of the disclosure. Saffron red powder is a natural carotenoid-containing compound. Saffron is a spice derived from Crocus sativus. It is characterized by a bitter taste and iodoform or hay-like fragrance; these are caused by the compounds picrocrocin and saffranal. It also contains the carotenoid dye crocin that gives its characteristic yellow-red color. Chlorophyll dyes—Examples of chlorophyll dyes that are useful in the compositions, methods, and uses of the disclosure, include but are not limited to chlorophyll a, chlorophyll b, oil soluble chlorophyll, bacteriochlorophyll a, bacteriochlorophyll b, bacteriochlorophyll c, bacteriochlorophyll d, protochlorophyll, protochlorophyll a, amphiphilic chlorophyll derivative 1, and amphiphilic chlorophyll derivative 2. In some aspects of the disclosure, the one or more chromophores of the composition disclosed herein can be independently selected from any of Acid black 1, Acid blue 22, Acid blue 93, Acid fuchsin, Acid green, Acid green 1, Acid green 5, Acid magenta, Acid orange 10, Acid red 26, Acid red 29, Acid red 44, Acid red 51, Acid red 66, Acid red 87, Acid red 91, Acid red 92, Acid red 94, Acid red 101, Acid red 103, Acid roseine, Acid rubin, Acid violet 19, Acid yellow 1, Acid yellow 9, Acid yellow 23, Acid yellow 24, Acid yellow 36, Acid yellow 73, Acid yellow S, Acridine orange, Acriflavine, Alcian blue, Alcian yellow, Alcohol soluble eosin, Alizarin, Alizarin blue 2RC, Alizarin carmine, Alizarin cyanin BBS, Alizarol cyanin R, Alizarin red S, Alizarin purpurin, Aluminon, Amido black 10B, Amidoschwarz, Aniline blue WS, Anthracene blue SWR, Auramine O, Azocannine B, Azocarmine G, Azoic diazo 5, Azoic diazo 48, Azure A, Azure B, Azure C, Basic blue 8, Basic blue 9, Basic blue 12, Basic blue 15, Basic blue 17, Basic blue 20, Basic blue 26, Basic brown 1, Basic fuchsin, Basic green 4, Basic orange 14, Basic red 2 (Safranin O), Basic red 5, Basic red 9, Basic violet 2, Basic violet 3, Basic violet 4, Basic violet 10, Basic violet 14, Basic yellow 1, Basic yellow 2, Biebrich scarlet, Bismarck brown Y, Brilliant crystal scarlet 6R, Calcium red, Carmine, Carminic acid (acid red 4), Celestine blue B, China blue, Cochineal, Celestine blue, Chrome violet CG, Chromotrope 2R, Chromoxane cyanin R, Congo corinth, Congo red, Cotton blue, Cotton red, Croceine scarlet, Crocin, Crystal ponceau 6R, Crystal violet, Dahlia, Diamond green B, DiOC6, Direct blue 14, Direct blue 58, Direct red, Direct red 10, Direct red 28, Direct red 80, Direct yellow 7, Eosin B, Eosin Bluish, Eosin, Eosin Y, Eosin yellowish, Eosinol, Erie garnet B, Eriochrome cyanin R, Erythrosin B, Ethyl eosin, Ethyl green, Ethyl violet, Evans blue, Fast blue B, Fast green FCF, Fast red B, Fast yellow, Fluorescein, Food green 3, Gallein, Gallamine blue, Gallocyanin, Gentian violet, Haematein, Haematine, Haematoxylin, Helio fast rubin BBL, Helvetia blue, Hematein, Hematine, Hematoxylin, Hoffman's violet, Imperial red, Indocyanin green, Ingrain blue, Ingrain blue 1, Ingrain yellow 1, INT, Kermes, Kermesic acid, Kernechtrot, Lac, Laccaic acid, Lauth's violet, Light green, Lissamine green SF, Luxol fast blue, Magenta 0, Magenta I, Magenta II, Magenta III, Malachite green, Manchester brown, Martius yellow, Merbromin, Mercurochrome, Metanil yellow, Methylene azure A, Methylene azure B, Methylene azure C, Methylene blue, Methyl blue, Methyl green, Methyl violet, Methyl violet 2B, Methyl violet 10B, Mordant blue 3, Mordant blue 10, Mordant blue 14, Mordant blue 23, Mordant blue 32, Mordant blue 45, Mordant red 3, Mordant red 11, Mordant violet 25, Mordant violet 39 Naphthol blue black, Naphthol green B, Naphthol yellow S, Natural black 1, Natural red, Natural red 3, Natural red 4, Natural red 8, Natural red 16, Natural red 25, Natural red 28, Natural yellow 6, NBT, Neutral red, New fuchsin, Niagara blue 3B, Night blue, Nile blue, Nile blue A, Nile blue oxazone, Nile blue sulphate, Nile red, Nitro BT, Nitro blue tetrazolium, Nuclear fast red, Oil red 0, Orange G, Orcein, Pararosanilin, Phloxine B, phycobilins, Phycocyanins, Phycoerythrins. Phycoerythrincyanin (PEC), Phthalocyanines, Picric acid, Ponceau 2R, Ponceau 6R, Ponceau B, Ponceau de Xylidine, Ponceau S, Primula, Purpurin, Pyronin B, Pyronin G, Pyronin Y, Rhodamine B, Rosanilin, Rose bengal, Saffron, Safranin O, Scarlet R, Scarlet red, Scharlach R, Shellac, Sirius red F3B, Solochrome cyanin R, Soluble blue, Solvent black 3, Solvent blue 38, Solvent red 23, Solvent red 24, Solvent red 27, Solvent red 45, Solvent yellow 94, Spirit soluble eosin, Sudan III, Sudan IV, Sudan black B, Sulfur yellow S, Swiss blue, Tartrazine, Thioflavine S, Thioflavine T, Thionin, Toluidine blue, Toluyline red, Tropaeolin G, Trypaflavine, Trypan blue, Uranin, Victoria blue 4R, Victoria blue B, Victoria green B, Water blue I, Water soluble eosin, Xylidine ponceau, or Yellowish eosin.

In some embodiments, the composition includes Eosin Y as a first light-absorbing molecule. In some embodiments, the composition includes Eosin Y as a first light-absorbing molecule and any one or more of Rose Bengal, Fluorescein, Erythrosin, Phloxine B as a second light-absorbing molecule.

Other suitable light-absorbing molecules that can be used in the composition of the present technology are endogenous light-absorbing molecules such as, but not limited to, vitamins. Examples of vitamins that may act as endogenous light-absorbing molecules include, vitamin B. In some instances, the endogenous light-absorbing molecule is vitamin B12. In some instances, the endogenous light-absorbing molecule is 7,8-Dimethyl-10-[(2S,3S,4R)-2,3,4,5-tetrahydroxypentyl]benzo[g]pteridine-2,4-dione.

In some embodiments, the composition includes the following synergistic combinations: Eosin Y and Fluorescein; Fluorescein and Rose Bengal; Erythrosine in combination with one or more of Eosin Y, Rose Bengal or Fluorescein; or Phloxine B in combination with one or more of Eosin Y, Rose Bengal, Fluorescein and Erythrosine. Other synergistic light-absorbing molecule combinations are also possible.

By means of synergistic effects of the light-absorbing molecule combinations in the composition, light-absorbing molecules which cannot normally be activated by an activating light (such as a blue light from an LED) can be activated through energy transfer from the light-absorbing molecules which are activated by the activating light. In this way, the different properties of photoactivated light-absorbing molecules can be harnessed and tailored according to the therapy required.

Light-absorbing molecule combinations can also have a synergistic effect in terms of their photoactivated state. For example, two light-absorbing molecules may be used, one of which emits fluorescent light when activated in the blue and green range, and the other which emits fluorescent light in the red, orange and yellow range, thereby complementing each other and irradiating the target tissue with a broad wavelength of light having different depths of penetration into target tissue and different therapeutic effects.

In some embodiments, the present disclosure provides compositions that comprise at least a first light-absorbing molecule and a gelling agent. A gelling agent may comprise any ingredient suitable for use in a topical composition as described herein. The gelling agent may be an agent capable of forming a cross-linked matrix, including physical and/or chemical cross-links. The gelling agent is preferably biocompatible, and may be biodegradable. In some implementations, the gelling agent is able to form a hydrogel or a hydrocolloid. An appropriate gelling agent is one that can form a viscous liquid or a semisolid. In preferred embodiments, the gelling agent and/or the composition has an appropriate light transmission property. The gelling agent preferably allows activity of the light-absorbing molecule(s). For example, some light-absorbing molecules require a hydrated environment in order to fluoresce. The gelling agent may be able to form a gel by itself or in combination with other ingredients such as water or another gelling agent, or when applied to a treatment site, or when illuminated with light.

The gelling agent according to various embodiments of the present disclosure may include, but not be limited to, polyalkylene oxides, particularly polyethylene glycol and poly(ethylene oxide)-poly(propylene oxide) copolymers, including block and random copolymers; polyols such as glycerol, polyglycerol (particularly highly branched polyglycerol), propylene glycol and trimethylene glycol substituted with one or more polyalkylene oxides, e.g., mono-, di- and tri-polyoxyethylated glycerol, mono- and di-polyoxy-ethylated propylene glycol, and mono- and di-polyoxyethylated trimethylene glycol; polyoxyethylated sorbitol, polyoxyethylated glucose; acrylic acid polymers and analogs and copolymers thereof, such as polyacrylic acid per se, polymethacrylic acid, poly(hydroxyethylmethacrylate), poly(hydroxyethylacrylate), poly(methylalkylsulfoxide methacrylate), poly(methylalkylsulfoxide acrylate) and copolymers of any of the foregoing, and/or with additional acrylate species such as aminoethyl acrylate and mono-2-(acryloxy)-ethyl succinate; polymaleic acid; poly(acrylamides) such as polyacrylamide per se, poly(methacrylamide), poly(dimethylacrylamide), and poly(N-isopropyl-acrylamide); poly(olefinic alcohol)s such as poly(vinyl alcohol); poly(N-vinyl lactams) such as poly(vinyl pyrrolidone), poly(N-vinyl caprolactam), and copolymers thereof, polyoxazolines, including poly(methyloxazoline) and poly(ethyloxazoline); and polyvinylamines

In some embodiments, the gelling agent comprises a carbomer. Carbomers are synthetic high molecular weight polymer of acrylic acid that are cross-linked with either allylsucrose or allylethers of pentaerythritol having a molecular weight of about 3×10⁶. The gelation mechanism depends on neutralization of the carboxylic acid moiety to form a soluble salt. The polymer is hydrophilic and produces sparkling clear gels when neutralized. Carbomer gels possess good thermal stability in that gel viscosity and yield value are essentially unaffected by temperature. As a topical product, carbomer gels possess optimum rheological properties. The inherent pseudoplastic flow permits immediate recovery of viscosity when shear is terminated and the high yield value and quick break make it ideal for dispensing. Aqueous solution of Carbopol® is acidic in nature due to the presence of free carboxylic acid residues. Neutralization of this solution cross-links and gelatinizes the polymer to form a viscous integral structure of desired viscosity. Carbomers are available as fine white powders which disperse in water to form acidic colloidal suspensions (a 1% dispersion has a pH of approximately 3) of low viscosity. Neutralization of these suspensions using a base, for example sodium, potassium or ammonium hydroxides, low molecular weight amines and alkanolamines, results in the formation of translucent gels. Nicotine salts such as nicotine chloride form stable water-soluble complexes with carbomers at about pH 3.5 and are stabilized at an optimal pH of about 5.6.

In some implementations, the carbomer is Carbopol®. Such polymers are commercially available from B.F. Goodrich or Lubrizol under the designation Carbopol® 71G NF, 420, 430, 475, 488, 493, 910, 934, 934P, 940, 971PNF, 974P NF, 980 NF, 981 NF and the like. In some embodiments, the carbomer is Carbopol® 974P NF, 980 NF, 5984 EP, ETD 2020NF, Ultrez 10 NF, 934 NF, 934P NF or 940 NF. In some embodiments, the carbomer is Carbopol® 980 NF, ETD 2020 NF, Ultrez 10 NF, Ultrez 21 or 1382 Polymer, 1342 NF, 940 NF. In some embodiments, from about 0.05% to about 10%, about 0.5% to about 5%, or about 1% to about 3% by weight of the total composition of a high molecular weight carbopol can be present as the gelling agent. In some embodiments, the biophotonic composition of the disclosure comprises from about 0.05% to about 10%, about 0.5% to about 5%, or from about 1% to about 3% by weight of the total composition of a high molecular weight carbopol.

In some embodiments, the gelling agent comprises a hygroscopic and/or a hydrophilic material useful for their water attracting properties. The hygroscopic or hydrophilic material may include, but is not limited to, glucosamine, glucosamine sulfate, polysaccharides, cellulose derivatives (hydroxypropyl methylcellulose, hydroxyethyl cellulose, hydroxypropyl cellulose, methylcellulose and the like), noncellulose polysaccharides (galactomannans, guar gum, carob gum, gum arabic, sterculia gum, agar, alginates and the like), glycosaminoglycan, poly(vinyl alcohol), poly(2-hydroxyethylmethylacrylate), polyethylene oxide, collagen, chitosan, alginate, a poly(acrylonitrile)-based hydrogel, poly(ethylene glycol)/poly(acrylic acid) interpenetrating polymer network hydrogel, polyethylene oxide-polybutylene terephthalate, hyaluronic acid, high-molecular-weight polyacrylic acid, poly(hydroxy ethylmethacrylate), poly(ethylene glycol), tetraethylene glycol diacrylate, polyethylene glycol methacrylate, and poly(methyl acrylate-co-hydroxyethyl acrylate). In some embodiments, the hydrophilic gelling agent is selected from glucose, modified starch, methyl cellulose, carboxymethyl cellulose, propyl cellulose, hydroxypropyl cellulose, carbomers, alginic acid, sodium alginate, potassium alginate, ammonium alginate, calcium alginate, agar, carrageenan, locust bean gum, pectin, and gelatin.

The gelling agent may be protein-based/naturally derived material such as sodium hyaluronate, gelatin or collagen, lipids, or the like. The gelling agent may be a polysaccharide such as starch, chitosan, chitin, agarose, agar, locust bean gum, carrageenan, gellan gum, pectin, alginate, xanthan, guar gum, and the like.

In some embodiments, the composition can include up to about 2% by weight of the final composition of sodium hyaluronate as the single gelling agent. In some embodiments, the composition can include more than about 4% or more than about 5% by weight of the total composition of gelatin as the single gelling agent. In some embodiments, the composition can include up to about 10% or up to about 8% starch as the single gelling agent. In some embodiments, the composition can include more than about 5% or more than about 10% by weight of the total composition of collagen as the gelling agent. In some embodiments, about 0.1% to about 10% or about 0.5% to about 3% by weight of the total composition of chitin can be used as the gelling agent. In some embodiments, about 0.5% to about 5% by weight of the final composition of corn starch or about 5% to about 10% by weight of the total composition of corn starch can be used as the gelling agent. In some embodiments, more than about 2.5% by weight of the total composition of alginate can be used in the composition as the gelling agent. In some embodiments, the percentages by weight percent of the final composition of the gelling agents can be as follows: cellulose gel (from about 0.3% to about 2.0%), konjac gum (from about 0.5% to about 0.7%), carrageenan gum (from about 0.02% to about 2.0%), xanthan gum (from about 0.01% to about 2.0%), acacia gum (from about 3% to about 30%), agar (from about 0.04% to about 1.2%), guar gum (from about 0.1% to about 1%), locust bean gum (from about 0.15% to about 0.75%), pectin (from about 0.1% to about 0.6%), tara gum (from about 0.1% to about 1.0%), polyvinylypyrrolidone (from about 1% to about 5%), sodium polyacrylate (from about 1% to about 10%). Other gelling agents can be used in amounts sufficient to gel the composition or to sufficiently thicken the composition. It will be appreciated that lower amounts of the above gelling agents may be used in the presence of another gelling agent or a thickener.

In the compositions and methods of the present disclosure, additional components may optionally be included, or used in combination with the compositions as described herein. Such additional components include, but are not limited to, chelating agents, polyols, healing factors, growth factors, antimicrobials, wrinkle fillers (e.g. botox, hyaluronic acid or polylactic acid), collagen, anti-virals, anti-fungals, antibiotics, drugs, and/or agents that promote collagen synthesis. These additional components may be applied to the wound, skin or mucosa in a topical fashion, prior to, at the same time of, and/or after topical application of the composition of the present disclosure, and may also be systemically administered. Suitable healing factors, antimicrobials, collagen, and/or agents that promote collagen synthesis are discussed below:

Healing factors comprise compounds that promote or enhance the healing or regenerative process of the tissues on the application site of the composition. During the photoactivation of the composition of the present disclosure, there may be an increase of the absorption of molecules at the treatment site by the skin, wound or the mucosa. An augmentation in the blood flow at the site of treatment is observed for a period of time. An increase in the lymphatic drainage and a possible change in the osmotic equilibrium due to the dynamic interaction of the free radical cascades can be enhanced or even fortified with the inclusion of healing factors. Suitable healing factors include, but are not limited to: hyaluronic acid, glucosamine, allantoin, saffron.

Examples of antimicrobials (or antimicrobial agent) are recited in U.S. Patent Application Publication Nos: 2004/0009227 and 2011/0081530, which are both herein incorporated by reference. Suitable antimicrobials for use in the methods of the present disclosure include, but not limited to, phenolic and chlorinated phenolic and chlorinated phenolic compounds, resorcinol and its derivatives, bisphenolic compounds, benzoic esters (parabens), halogenated carbonilides, polymeric antimicrobial agents, thazolines, trichloromethylthioimides, natural antimicrobial agents (also referred to as “natural essential oils”), metal salts, and broad-spectrum antibiotics.

In some embodiments, the pH of the composition is in or adjusted to the range of about 4 to about 10. In some embodiments, the pH of the composition is in or adjusted to the range of about 4 to about 9. In some embodiments, the pH of the composition is in or adjusted to the range of about 4 to about 8. In some embodiments, the pH of the composition is within the range of about 4 to about 7. In some embodiments, the pH of the composition is within the range of about 4 to about 6.5. In some embodiments, the pH of the composition is within the range of about 4 to about 6. In some embodiments, the pH of the composition is within the range of about 4 to about 5.5. In some embodiments, the pH of the composition is within the range of about 4 to about 5. In some embodiments, the pH of the composition is within the range of about 5.0 to about 8.0. In some embodiments, the pH of the composition is within the range of about 6.0 to about 8.0. In some embodiments, the pH of the composition is within the range of about 6.5 to about 7.5. In some embodiments, the pH of the composition is within the range of about 5.5 to about 7.5.

In some embodiments, the pH of the composition is in or adjusted to the range of 4 to 10. In some embodiments, the pH of the composition is in or adjusted to the range of 4 to 9. In some embodiments, the pH of the composition is in or adjusted to the range of 4 to 8. In some embodiments, the pH of the composition is within the range of 4 to 7. In some embodiments, the pH of the composition is within the range of 4 to 6.5. In some embodiments, the pH of the composition is within the range of 4 to 6. In some embodiments, the pH of the composition is within the range of 4 to 5.5. In some embodiments, the pH of the composition is within the range of 4 to 5. In some embodiments, the pH of the composition is within the range of 5.0 to 8.0. In some embodiments, the pH of the composition is within the range of 6.0 to 8.0. In some embodiments, the pH of the composition is within the range of 6.5 to 7.5. In some embodiments, the pH of the composition is within the range of 5.5 to 7.5.

In some embodiments, the compositions of the disclosure also include an aqueous substance (water) or an alcohol. Alcohols include, but are not limited to, ethanol, propanol, isopropanol, butanol, iso-butanol, t-butanol or pentanol. In some embodiments, the chromophore or combination of chromophores is in solution in a medium of the composition. In some embodiments, the chromophore or combination of chromophores is in solution in a medium of the composition, wherein the medium is an aqueous substance.

The methods of the present disclosure are useful for enhancing hair growth, preventing and/or treating hair loss and for improving the quality of hair, such as improving luster, sheen, brilliance, gloss, glow, shine or patina of hair associated with the follicles.

In some instances, the methods of the present disclosure are used to prevent and/or treat various types of alopecia and other conditions associated with hair loss. Alopecia is the most common hair growth disorder in humans. Hair loss most commonly occurs from the scalp. However, any hair-bearing area can be affected, including eyebrows, eyelashes, beard, and body areas. Alopecia can be divided into disorders in which the hair follicle is normal but the cycling of hair growth is abnormal, and disorders in which the hair follicle is damaged. Six major types of alopecia are known: androgenic alopecia, alopecia areata, anagen effluvium, self-induced hair loss, telogen effluvium, and scarring alopecia.

Androgenetic alopecia includes male pattern baldness and female pattern baldness. Androgenetic alopecia accounts for 95% of all hair loss. This genetically determined disorder is progressive through the gradual conversion of large, thick, pigmented, terminal hair into thinner, shorter, indeterminate hair and finally to short, wispy, non-pigmented, vellus hair. Patients have a reduction in the terminal-to-vellus hair ratio, normally at least 2:1. Following miniaturization of the follicles, fibrous tracts remain Patients with this disorder usually have a typical distribution of hair loss. Male pattern alopecia begins with the recession of the hairline and results in complete hair loss, while female pattern alopecia causes diffuse thinning of the hair at and behind the hairline and there is no recession of the hairline. Male pattern alopecia begins in the late teens and early 20's when the testosterone levels are high, while female pattern alopecia begins in the late 30's and reaches its peak after 50 when testosterone levels are falling. Male pattern alopecia affects up to 70% of all males, whereas female pattern alopecia affects up to 30% of women. Females with predisposition for male pattern alopecia rapidly develop typical male pattern baldness if given high doses of testosterone.

Alopecia areata is thought to be an autoimmune disease in which T-lymphocytes attack the hair follicles, causing the hair to stop growing and enter into the telogen phase. At the end of the telogen phase, the hair falls out. Alopecia areata affects both men and women equally and is often experienced first in childhood. There are three subtypes of alopecia areata which are named according to their severity: (i) Alopecia areata, which involves mild patchy hair loss on the scalp; (ii) Alopecia totalis which involves loss of all scalp hair; and (iii) Alopecia universalis which involves loss of scalp and all body hair.

Anagen effluvium is the sudden hair loss, which occurs as a result of exposure to chemicals or radiation, such as the hair loss that results during certain types of chemotherapy or radiation treatment, or as a result of exposure to toxic chemicals such as thallium and arsenic. In anagen effluvium the hair does not enter a resting stage. The hair loss is usually sudden occurring 1 to 3 weeks after expose to the chemicals or radiation has occurred. In most cases hair growth will return to normal once treatment is finished. The drugs which are most likely to cause hair loss include amsacrine; cisplatinum; cytosine arabinoside; cyclophosphamide; doxorubicin; epirubicin; etoposide ifosfamide; and vincristine. It has been found that agents which protect against alopecia induced by a particular drug may be ineffective in protecting against a different drug. For example, a composition obtained from the bacteria Serratia marcescens has been used to protect against the alopecia which is associated with the use of cytosine araginoside and doxorubicin. This composition had no effect on alopecia which was induced by cyclophosphamide.

Self-induced hair loss may be inflicted consciously or unconsciously. The two main types of self-induced hair loss are trichotillomania and traction alopecia. Trichotillomania is self-induced hair loss which results from the continuous pulling or plucking of the hair. It occurs most commonly among young children, adolescents and women and affects twice as many females as males. The hair is often pulled out in distinct patches on the scalp. Some individuals also pull out eyebrows and eyelashes. The treatment for trichotillomania often involves counseling or psychiatric help, whereby in some cases an antidepressant is prescribed. Traction alopecia is usually caused by continuous and excessive pulling on the hair due to various types of hairstyling, which gradually results in hair loss that may become permanent. Generally, a change in hairstyle that reduces the traction on the hair and hair follicle is sufficient to reverse the hair loss in this case.

Telogen effluvium is sudden or severe stress related hair loss, which appears as thinning throughout the whole scalp. A sudden or stressful event can cause the hair follicles to prematurely stop growing and enter into a resting phase. The hair will then stay in the resting phase for about 3 months after which time a large amount of hair will be shed. In most cases the hair loss is temporary and the hair soon recovers. In some cases, the hair loss continues until the underlying cause is removed. Events which may lead to telogen effluvium include childbirth; termination of pregnancy; starting or stopping birth control pills; use of various medications; and severe emotional stress. Increased levels of hormones estrogen and progesterone during pregnancy cause more hair than normal to remain in the growth phase. Following childbirth or termination of pregnancy, many of the hair follicles that had delayed entering the resting phase suddenly enter the resting phase due to the rapid drop in hormone levels. Drugs which may cause hair loss as a side effect include anti-gout agents such as alloppurinol; blood thinners such as heparin and coumarin; and cholesterol lowering drugs such as clofibrate and gemfibrozil. Telogen effluvium may also occur after a traumatic event such as the death of a loved one, an accident, abuse or any other severely traumatic event. These events may trigger the hair follicles to enter the resting phase prematurely in which case an increase in the amount of hair shed will be noticed about 3 months after the event. Other causes of telogen effluvium include thyroid gland malfunction (hypothyroidism or hyperthyroidism, which occurs when the thyroid gland produces too little or too much, respectively, of the thyroid hormone, thyroxin); diabetes; anemia; and the autoimmune disease, systemic lupus erythematosis.

Scarring alopecia occurs as a result of inflammation of the hair follicles due to infection. Scarring alopecia may be caused by discoid lupus erythematosus, a diffuse connective tissue disease; lichenplanus, which is an inflammatory disease that strikes primarily the skin and mucous membranes; Pseudopelade of Brocq, a rare scarring alopecia which has no potential for regrowth; aplasia cutis congenita, a rare disorder that often results as a small blistered atrophied area usually in the midline of the scalp and present from birth; or congenital ctrichia. Other types of hair loss include syphilitic alopecia, a secondary manifestation of syphilis; scleroderma; and tinea capitis (ringworm).

In some embodiments, the methods of the present disclosure comprise applying a composition of the present disclosure to an area of the skin of a subject that is in need of hair growth and illuminating the applied composition with light having a wavelength that overlaps with an absorption spectrum of the at least one light-absorbing molecule of the composition. In some implementations, the composition is applied topically.

In the methods of the present disclosure, any source of actinic light can be used to illuminate the compositions. Any type of halogen, LED or plasma arc lamp or laser may be suitable. The primary characteristic of suitable sources of actinic light will be that they emit light in a wavelength (or wavelengths) appropriate for activating the one or more photoactivators present in the composition. In some instances, an argon laser is used. In some instances, a potassium-titanyl phosphate (KTP) laser (e.g., a GreenLight™ laser) is used. In other instances, sunlight may be used. In some instances, a LED photocuring device is the source of the actinic light. The source of the actinic light is a source of light having a wavelength between about 200 nm and about 800 nm, between about 400 nm and about 700 nm, between about 400 nm and about 600 nm, between about 400 nm and about 550 nm, between about 380 nm and about 700 nm, between about 380 nm and about 600 nm, between about 380 nm and about 550 nm, between about 200 nm and about 800 nm, between about 400 nm and about 700 nm, between about 400 nm and about 600 nm, between about 400 nm and about 550 nm, between about 380 nm and about 700 nm, between about 380 nm and about 600 nm, or between about 380 nm and about 550 nm. In some instances, the composition of the disclosure is illuminated with violet and/or blue light. Furthermore, the source of actinic light should have a suitable power density. Suitable power density for non-collimated light sources (LED, halogen or plasma lamps) are in the range from about 1 mW/cm² to about 1200 mW/cm², such as from about 20 mW/cm² to about 1000 mW/cm² from about 100 mW/cm² to about 900 mW/cm² from about 200 mW/cm² to about 800 mW/cm², or from about 1 mW/cm² to about 200 mW/cm². In some embodiments, the power density for non-collimated light sources (LED, halogen or plasma lamps) are in the range from about 1 mW/cm² to about 200 mW/cm² Suitable power density for laser light sources is in the range from about 0.5 mW/cm² to about 0.8 mW/cm².

In some embodiments of the methods of the present disclosure, the light has an energy at the subject's skin of from about 1 mW/cm² to about 500 mW/cm², or about 1 mW/cm² to about 300 mW/cm², or about 1 mW/cm² to about 200 mW/cm², wherein the energy applied depends at least on the condition being treated, the wavelength of the light, the distance of the subject's skin from the light source, and the thickness of the composition. In some embodiments, the light at the subject's skin is from about 1 mW/cm² to about 40 mW/cm², or about 20 mW/cm² to about 60 mW/cm², or about 40 mW/cm² to about 80 mW/cm², or about 60 mW/cm² to about 100 mW/cm², or about 80 mW/cm² to about 120 mW/cm², or about 100 mW/cm² to about 140 mW/cm², or about 120 mW/cm² to about 160 mW/cm², or about 140 mW/cm² to about 180 mW/cm², or about 160 mW/cm² to about 200 mW/cm², or about 110 mW/cm² to about 240 mW/cm², or about 110 mW/cm² to about 150 mW/cm², or about 190 mW/cm² to about 240 mW/cm². In certain embodiments, the fluence delivered to the treatment areas may be between about 1 to about 60 J/cm², about 4 to about 60 J/cm², about 10 to about 60 J/cm², about 10 to about 50 J/cm², about 10 to about 40 J/cm², about 10 to about 30 J/cm², about 20 to about 40 J/cm², about 15 J/cm² to 25 J/cm², or about 10 to about 20 J/cm².

In some embodiments, the light-activating molecule can be photoactivated by ambient light which may originate from the sun or other light sources. Ambient light can be considered to be a general illumination that comes from all directions in a room that has no visible source. The light-activating molecule can be photoactivated by light in the visible range of the electromagnetic spectrum. Exposure times to ambient light may be longer than that to direct light.

In some embodiments, different sources of light can be used to activate the compositions, such as a combination of ambient light and direct LED light.

The duration of the exposure to actinic light required will be dependent on the surface of the treated area, the severity of the condition that is being treated, the power density, wavelength and bandwidth of the light source, the thickness of the composition, and the treatment distance from the light source. The illumination of the treated area by fluorescence may take place within seconds or even fragment of seconds, but a prolonged exposure period is beneficial to exploit the synergistic effects of the absorbed, reflected and reemitted light on the composition of the present disclosure and its interaction with the tissue being treated. In some embodiments, the time of exposure to actinic light of the tissue or skin which the composition has been applied is a period from about 1 second to about 30 minutes, from about 1 minute to about 30 minutes, from about 1 minute to about 5 minutes, from about 1 minute to about 5 minutes, from about 20 seconds to about 5 minutes, from about 60 seconds to about 5 minutes, or for less than about 5 minutes, or between about 20 seconds to about 5 minutes, or from about about 60 seconds to about 5 minutes per cm² of the area to be treated, so that the total time of exposure of a 10 cm² area would be from about 10 minutes to about 50 minutes.

In some embodiments, the composition is illuminated for a period from about 1 minute and 3 minutes. In some embodiments, light is applied for a period of from about 1 second to about 30 seconds, from about 1 second to about 60 seconds, from about 15 seconds to about 45 seconds, from about 30 seconds to about 60 seconds, from about 0.75 minute to about 1.5 minutes, from about 1 minute to about 2 minutes, from about 1.5 minutes to about 2.5 minutes, from about 2 minutes to about 3 minutes, from about 2.5 minutes to about 3.5 minutes, from about 3 minutes to about 4 minutes, from about 3.5 minutes to about 4.5 minutes, from about 4 minutes to about 5 minutes, from about 5 minutes to about 10 minutes, from about 10 minutes to about 15 minutes, from about 15 minutes to about 20 minutes, from about 20 minutes to about 25 minutes, or from about 20 minutes to about 30 minutes. In some embodiments, light is applied for a period of 1 second, about 5 seconds, about 10 seconds, about 20 seconds, about 30 seconds, less than about 30 minutes, less than about 20 minutes, less than about 15 minutes, less than about 10 minutes, less than about 5 minutes, less than about 1 minute, less than about 30 seconds, less than about 20 seconds, less than 10 seconds, less than 5 seconds, or for less than 1 second.

In some embodiments, the source of actinic light is in continuous motion over the treated area for the appropriate time of exposure. In some instances, multiple applications of the composition and actinic light are performed. In some instances, the tissue or skin is exposed to actinic light at least two, three, four, five or six times. In some embodiments, the tissue or skin is exposed to actinic light at least two, three, four, five or six times with a resting period in between each exposure. In certain such embodiments, the resting period is less than about 1 minute, less than about 5 minutes, less than about 10 minutes, less than about 20 minutes, less about 40 minutes, less than about 60 minutes, less than about 2 hours, less than about 4 hours, less than about 6 hours, or less than 12 hours. In some embodiments, the entire treatment may be repeated in its entirety as may be required by the patient. In some embodiments, a fresh application of the composition is applied before another exposure to actinic light.

In the methods of the present disclosure, the composition may be optionally removed from the site of treatment following application of light. In some instances, the composition is left on the treatment site for more than about 30 minutes, more than one hour, more than about 2 hours, or more than about 3 hours. In some instances, the composition is left for a prolonged period of time such as, for example, over night. It can be illuminated with ambient light. To prevent drying, the composition can be covered with a transparent or translucent cover such as a polymer film, or an opaque cover which can be removed before illumination.

The compositions of the disclosure may be applied at regular intervals such as once a week. The compositions of the disclosure may be applied once per week for one or more weeks, such as once per week for one week. The compositions of the disclosure may be applied once per week for two weeks, once per week for three weeks, once per week for four weeks, once per week for five weeks, once per week for six weeks, once per week for seven weeks, or once per week for eight or more weeks.

The compositions of the disclosure may be applied twice per week for one or more weeks, such as twice per week for one week. The compositions of the disclosure may be applied twice per week for two weeks, twice per week for three weeks, twice per week for four weeks, twice per week for five weeks, twice per week for six weeks, twice per week for seven weeks, or twice per week for eight or more weeks.

The compositions of the disclosure may be applied three times or more per week for one or more weeks, such as three times or more for one week. The compositions of the disclosure may be applied three times or more per week for two weeks, three times or more per week for three weeks, three times or more per week for four weeks, three times or more per week for five weeks, three times or more per week for six weeks, three times or more per week for seven weeks, or three times or more per week for eight or more weeks.

In any of the above mentioned methods for monitoring hair loss, hair growth, and response to treatment, an improvement in hair growth or a positive response to treatment is determined as increase in hair density, increase in terminal hair density, vellus hair density or cumulative hair thickness, increase in anagen hair count, decrease in telogen hair count, increase in total hair count, and increase in linear hair growth rate.

In some embodiments, the compositions of the present disclosure may also be used in combination with compounds known to promote hair growth that are available such as, but not limited to, drugs, such as finasteride (Propecia), a type 2 5-alpha-reductase inhibitor, and dutasteride, a type 1- and 2-5-alpha-reductase inhibitor, as well as flutamide, bicalutamide, pregnane derivatives, progesterone derivatives, experimental agents, such as FCE 28260, and the like. Spironolactone and other diuretics may also be utilized as it is indicated for women in some cases (also known as Aldactone: an aldosterone receptor antagonist). Potassium channel openers, such as Minoxidil (Rogaine), which are known to promote hair growth are also believed to be especially promising combinations. Herbal remedies that may have 5-alpha reductase inhibitory action may include: Saw Palmetto and Pygeum africanum. Other agents that may have such activity are Beta-sisterol, Sepicontrol and Licorice, gamma-linolenic acid, zinc and zinc salts, green tea catechin (--)-epigallocatechin gallate (EGCG), and other unsaturated fatty acids. Grape seed, apple seed, apple juice and barley extracts may also be potential agents that may induce hair growth.

Additional combinations may include other known stimulators of hair growth, such as zinc, calcineurin inhibitors, such as FK506 (Tacrolimus, Fujimycin), a macrolide antibiotic produced by Streptomyces tsukubaensis, and its derivatives, or Cyclosporin A, a cyclic endecapeptide, alprostadil, latanoprost, and a T cell-specific immunosuppressant, and the like.

The present disclosure also provides kits for stimulating hair growth. In particular, the kit is for preparing and/or applying any of the compositions of the present disclosure to an area of the skin that is in need of enhancement of hair growth. The kit may include a composition, as defined herein, together with one or more of a light source, devices for applying or removing the composition, instructions of use for the composition and/or light source.

In some embodiments, the composition comprises at least a first light-absorbing molecule in a gelling agent. The light-absorbing molecule may be present in an amount of between about 0.001% and about 0.1%, between about 0.05% and about 1%, between about 0.5% and about 2%, between about 1% and about 5%, between about 2.5% and about 7.5%, between about 5% and about 10%, between about 7.5% and about 12.5%, between about 10% and about 15%, between about 12.5% and about 17.5%, between about 15% and about 20%, between about 17.5% and about 22.5%, between about 20% and about 25%, between about 22.5% and about 27.5%, between about 25% and about 30%, between about 27.5% and about 32.5%, between about 30% and about 35%, between about 32.5% and about 37.5%, or between about 35% and about 40% per weight of the composition. In embodiments where the composition comprises more than one light-absorbing molecule, the first light-absorbing molecule may be present in an amount of between about 0.01% and about 40% per weight of the composition, and a second light-absorbing molecule may be present in an amount of between about 0.0001% and about 40% per weight of the composition.

In certain embodiments, the first light-absorbing molecule is present in an amount of between about 0.01%-0.1%, between about 0.05%-1%, between about 0.5%-2%, between about 1%-5%, between about 2.5%-7.5%, between about 5%-10%, between about 7.5%-12.5%, between about 10%-15%, between about 12.5%-17.5%, between about 15%-20%, between about 17.5%-22.5%, between about 20%-25%, between about 22.5%-27.5%, between about 25%-30%, between about 27.5%-32.5%, between about 30%-35%, between about 32.5%-37.5%, or between about 35%-40% per weight of the composition. In certain embodiments, the second light-absorbing molecule is present in an amount of between about 0.001%-0.1%, between about 0.05%-1%, between about 0.5%-2%, between about 1%-5%, between about 2.5%-7.5%, between about 5%-10%, between about 7.5%-12.5%, between about 10%-15%, between about 12.5%-17.5%, between about 15%-20%, between about 17.5%-22.5%, between about 20%-25%, between about 22.5%-27.5%, between about 25%-30%, between about 27.5%-32.5%, between about 30%-35%, between about 32.5%-37.5%, or between about 35%-40% per weight of the composition. In certain embodiments, the amount of light-absorbing molecule or combination of light-absorbing molecules may be in the amount of between about 0.05%-40.05% per weight of the composition. In certain embodiments, the amount of light-absorbing molecule or combination of light-absorbing molecules may be in the amount of between about 0.001%-0.1%, between about 0.05%-1%, between about 0.5%-2%, between about 1%-5%, between about 2.5%-7.5%, between about 5%-10%, between about 7.5%-12.5%, between about 10%-15%, between about 12.5%-17.5%, between about 15%-20%, between about 17.5%-22.5%, between about 20%-25%, between about 22.5%-27.5%, between about 25%-30%, between about 27.5%-32.5%, between about 30%-35%, between about 32.5%-37.5%, or between about 35%-40.05% per weight of the composition. The composition may include an oxygen-releasing agent present in amount between about 0.01%-40%, between about 0.01%-1.0%, between about 0.5%-10.0%, between about 5%-15%, between about 10%-20%, between about 15%-25%, between about 20%-30%, between about 15.0%-25%, between about 20%-30%, between about 25%-35%, or between about 30%-40% by weight to weight of the composition. Alternatively, the kit may include the oxygen-releasing agent as a separate component to the light-absorbing molecule containing composition.

In some embodiments, the kit includes more than one composition, for example, a first and a second composition. The first composition may include the oxygen-releasing agent and the second composition may include the first light-absorbing molecule in the gelling agent. The first light-absorbing molecule may have an emission wavelength between about 400 nm and about 570 nm. The oxygen-releasing agent may be present in the first composition in an amount of between about 0.01%-1.0%, between about 0.5%-10.0%, between about 5%-15%, between about 10%-20%, between about 15%-25%, between about 20%-30%, between about 15.0%-25%, between about 20%-30%, between about 25%-35%, between about 30%-40% or between about 35%-45% by weight to weight of the first composition. The light-absorbing molecule may be present in the second composition in an amount of between about 0.001%-0.1%, between about 0.05%-1%, between about 0.5%-2%, between about 1%-5%, between about 2.5%-7.5%, between about 5%-10%, between about 7.5%-12.5%, between about 10%-15%, between about 12.5%-17.5%, between about 15%-20%, between about 17.5%-22.5%, between about 20%-25%, between about 22.5%-27.5%, between about 25%-30%, between about 27.5%-32.5%, between about 30%-35%, between about 32.5%-37.5%, or between about 35%-40% per weight of the second composition. In embodiments where the second composition comprises more than one light-absorbing molecule, the first light-absorbing molecule may be present in an amount of between about 0.01%-40% per weight of the second composition, and a second light-absorbing molecule may be present in an amount of about 0.0001%-40% per weight of the second composition. In certain embodiments, the first light-absorbing molecule is present in an amount of between about 0.001%-0.1%, between about 0.05%-1%, between about 0.5%-2%, between about 1%-5%, between about 2.5%-7.5%, between about 5%-10%, between about 7.5%-12.5%, between about 10%-15%, between about 12.5%-17.5%, between about 15%-20%, between about 17.5%-22.5%, between about 20%-25%, between about 22.5%-27.5%, between about 25%-30%, between about 27.5%-32.5%, between about 30%-35%, between about 32.5%-37.5%, or between about 35%-40% per weight of the second composition. In certain embodiments, the second light-absorbing molecule is present in an amount of between about 0.001%-0.1%, between about 0.05%-1%, between about 0.5%-2%, between about 1%-5%, between about 2.5%-7.5%, between about 5%-10%, between about 7.5%-12.5%, between about 10%-15%, between about 12.5%-17.5%, between about 15%-20%, between about 17.5%-22.5%, between about 20%-25%, between about 22.5%-27.5%, between about 25%-30%, between about 27.5%-32.5%, between about 30%-35%, between about 32.5%-37.5%, or between about 35%-40% per weight of the second composition. In certain embodiments, the amount of light-absorbing molecule or combination of light-absorbing molecules may be in the amount of about 0.05%-40.05% per weight of the second composition. In certain embodiments, the amount of light-absorbing molecule or combination of light-absorbing molecules may be in the amount of between about 0.001%-0.1%, between about 0.05%-1%, between about 0.5%-2%, between about 1%-5%, between about 2.5%-7.5%, between about 5%-10%, between about 7.5%-12.5%, between about 10%-15%, between about 12.5%-17.5%, between about 15%-20%, between about 17.5%-22.5%, between about 20%-25%, between about 22.5%-27.5%, between about 25%-30%, between about 27.5%-32.5%, between about 30%-35%, between about 32.5%-37.5%, or between about 35%-40.05% per weight of the second light-absorbing molecule.

In some other embodiments, the first composition may comprise the first light-absorbing molecule in a liquid or as a powder, and the second composition may comprise a gelling composition for thickening the first composition. The oxygen-releasing agent may be contained in the second composition or in a third composition in the kit. In some embodiments, the kit includes containers comprising the compositions of the present disclosure. In some embodiments, the kit includes a first container comprising a first composition that includes the oxygen-releasing agent, and a second container comprising a second composition that includes at least one light-absorbing molecule. The containers may be light impermeable, air-tight and/or leak resistant. Exemplary containers include, but are not limited to, syringes, vials, or pouches. The first and second compositions may be included within the same container but separated from one another until a user mixes the compositions. For example, the container may be a dual-chamber syringe where the contents of the chambers mix on expulsion of the compositions from the chambers. In another example, the pouch may include two chambers separated by a frangible membrane. In another example, one component may be contained in a syringe and injectable into a container comprising the second component.

The composition may also be provided in a container comprising one or more chambers for holding one or more components of the composition, and an outlet in communication with the one or more chambers for discharging the composition from the container.

In some embodiments, the kit comprises a systemic or topical drug for augmenting the treatment of the composition. For example, in certain such embodiments, the kit may include a systemic or topical agent, e.g., an anesthetics or anti-inflammation agent, for reducing pain.

Written instructions on how to use the composition in accordance with the present disclosure may be included in the kit, or may be included on or associated with the containers comprising the compositions of the present disclosure.

In certain embodiments, the kit may comprise a further component which is a dressing. The dressing may be a porous or semi-porous structure for receiving the composition. The dressing may comprise woven or non-woven fibrous materials.

In certain embodiments of the kit, the kit may further comprise a light source such as a portable light with a wavelength appropriate to activate the light-absorbing molecule in the composition. The portable light may be battery operated or re-chargeable.

In certain embodiments, the kit may further comprise one or more waveguides.

Identification of equivalent compositions, methods and kits are well within the skill of the ordinary practitioner and would require no more than routine experimentation, in light of the teachings of the present disclosure. Practice of the disclosure will be still more fully understood from the following examples, which are presented herein for illustration only and should not be construed as limiting the disclosure in any way.

### EXAMPLES

The examples below are given so as to illustrate the practice of various embodiments of the present disclosure. They are not intended to limit or define the entire scope of this disclosure. It should be appreciated that the disclosure is not limited to the particular embodiments described and illustrated herein but includes all modifications and variations falling within the scope of the disclosure as defined in the appended embodiments.

The biophotonic compositions and methods of the present disclosure (biophotonic therapy) were tested to analyze the enhancement of hair growth on wounded skin for areas treated with biophotonic therapy compared to areas that were not treated with the biophotonic therapy. Subjects 1 and 2 had simple surgical wounds from orthopedic surgeries, whereas subjects 3, 4 and 5 had traumatic ulcers. From the first day after operation (T0) and then every 3 days for 14 days, 50% of the length of the surgical wound was treated with biophotonic therapy using a biophotonic composition comprising a carrier gel comprising peroxide in the form of urea peroxide (UP) (6% UP) and a light-absorbing molecule-containing gel (final eosin Y concentration of 0.01% w/w), while 50% % of the length of the surgical wound was treated with sterile saline. In cases where the subjects exhibited two surgical wounds (common in joint diseases often congenital and bilateral), one was treated with the biophotonic composition and the other treated daily with sterile saline. The subjects that were treated with the biophotonic therapy every 3 days until healing occurred. The biophotonic therapy included an illumination period of 2 minutes. Every three days, the treatment area was cleaned with sterile isotonic saline and then treated with the biophotonic composition. After treatment, the wound was cleaned and then covered with a three-layer bandage. The therapy was suspended after complete healing of the wound. Samples of skin from the areas treated with the biophotonic therapy and from the areas that were not treated with the biophotonic therapy were then assessed for expression of various cellular markers associated with the process of skin healing as well as for counts of anagenic, telogenic and catagenic follicules. Table 1 outlines the various cellular markers that were assessed.

TABLE 1
Cellular markers

| Marker | Indicator of: |
|---|---|
| TNFalpha | Inflammation |
| TGFbeta | Tissue repair |
| FVIII | Tissue repair |
| EGF | Epidermis condition |
| FGF | Dermis condition |
| Decorin | Fibrillogenesis, angiogenesis |
| Collagen III | Tissue granulation |
| Hsp60 | Mitochondrial integrity and well preservation |
| Ki67 | Cellular division associated to energetic levels |

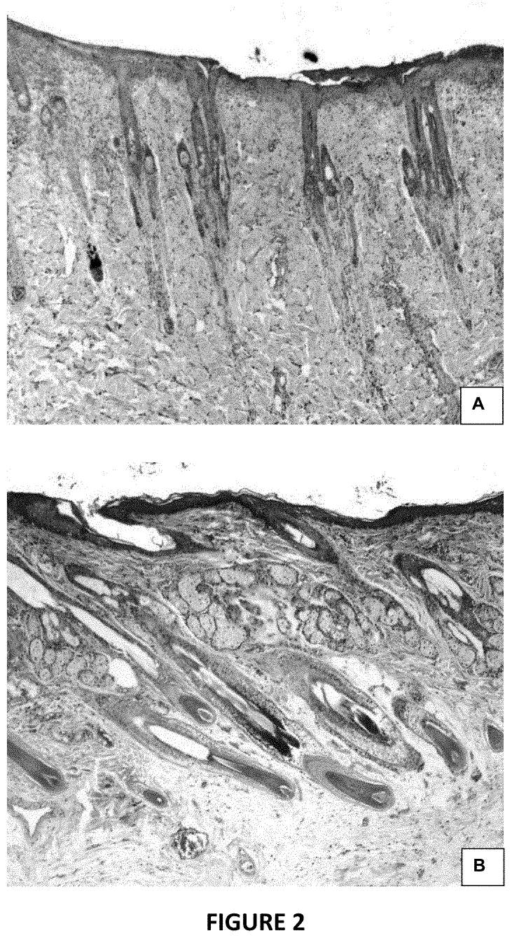

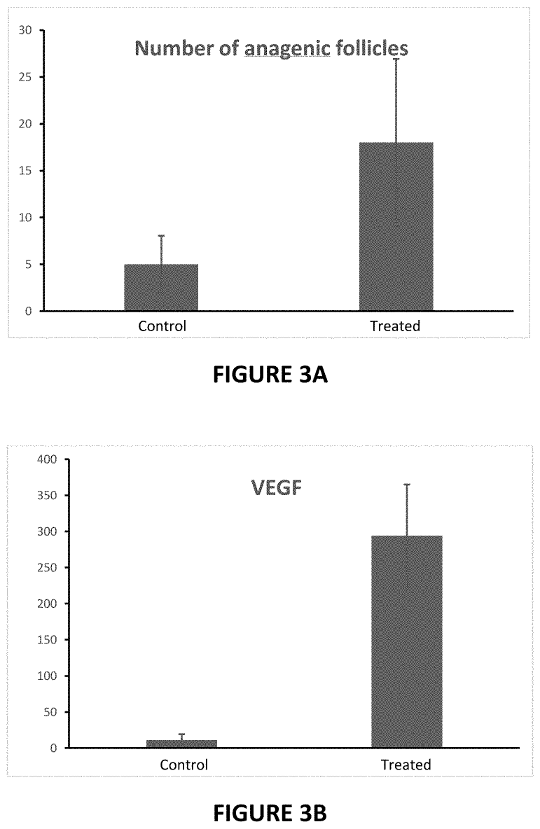

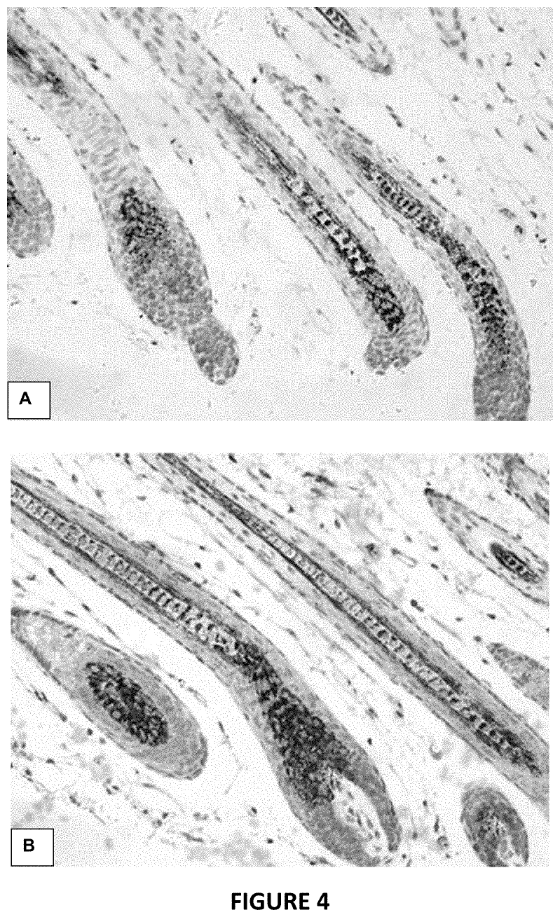

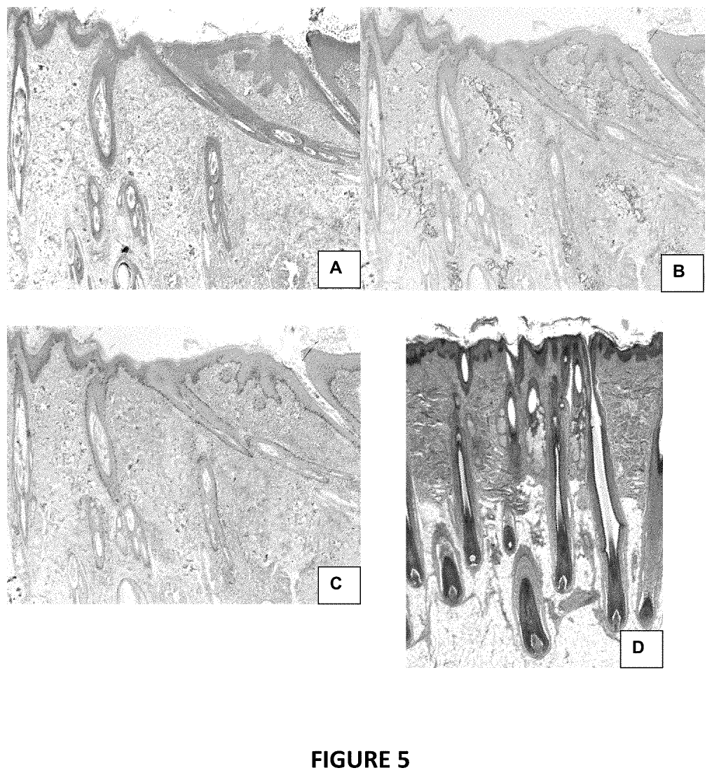

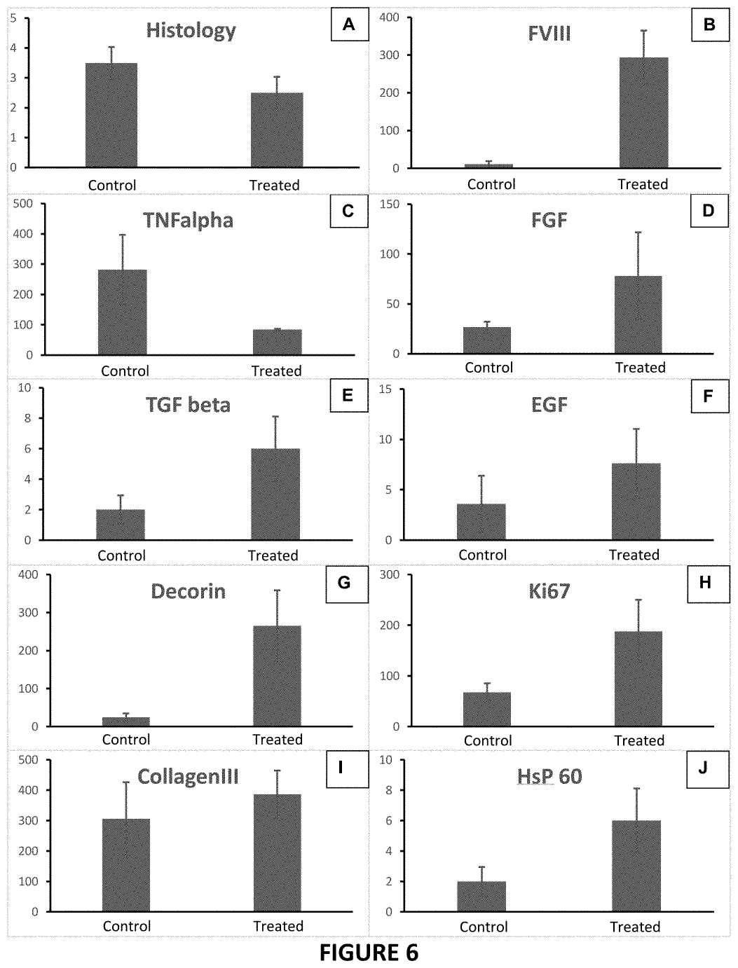

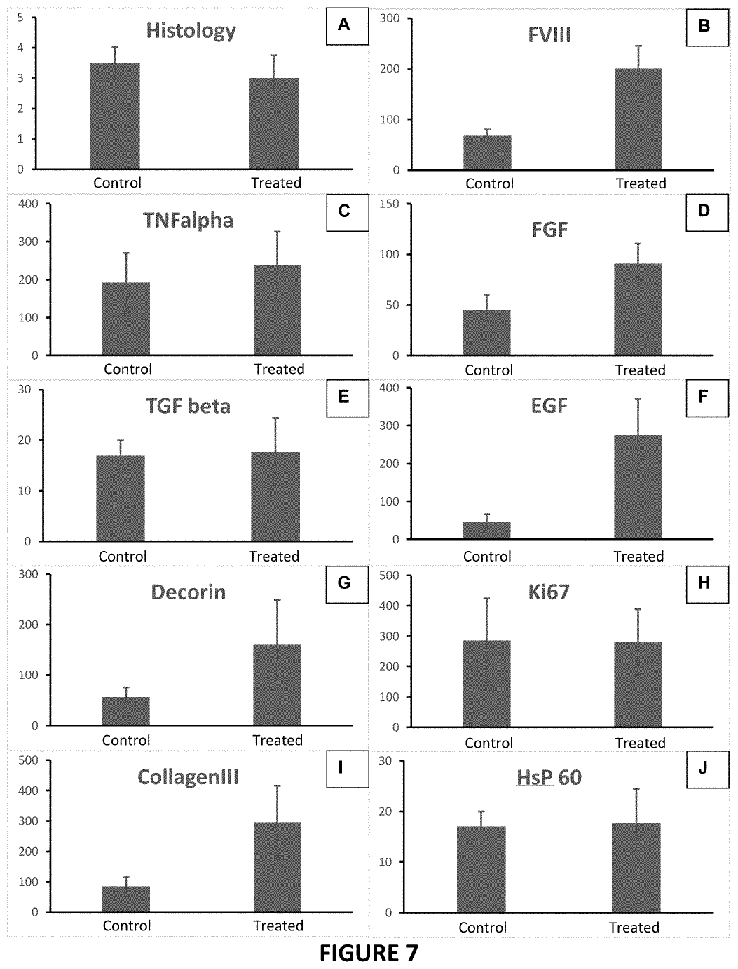

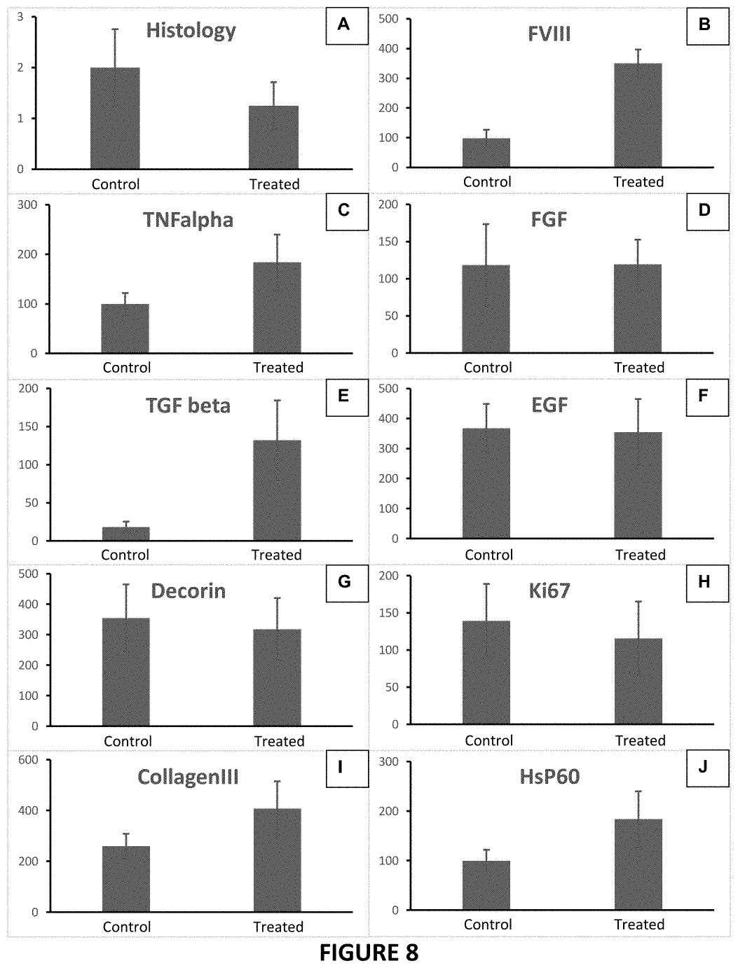

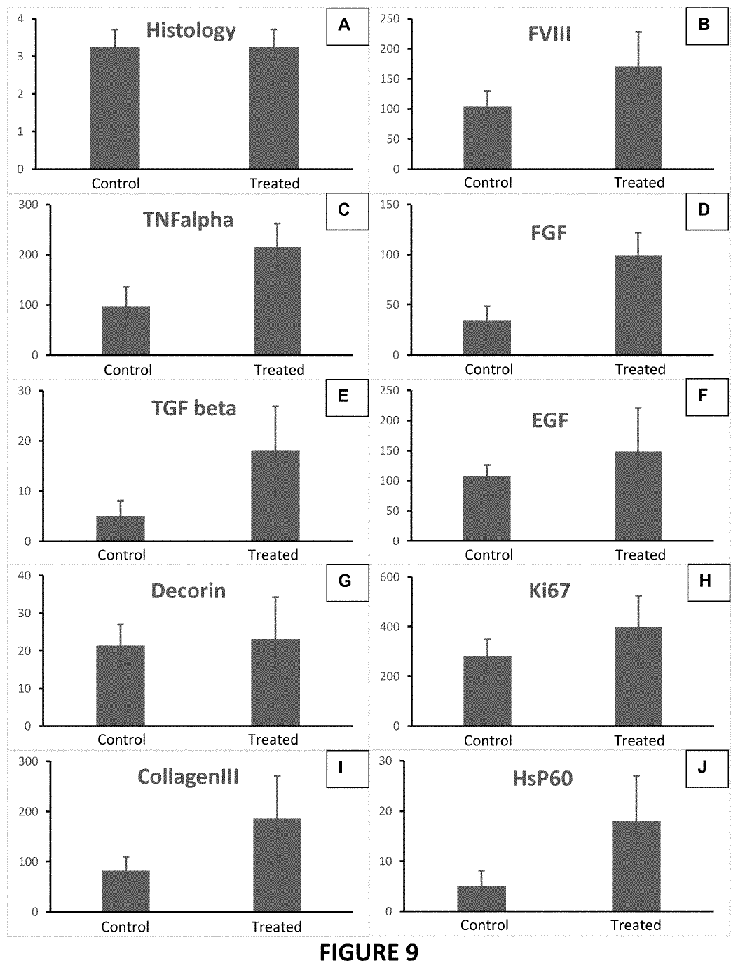

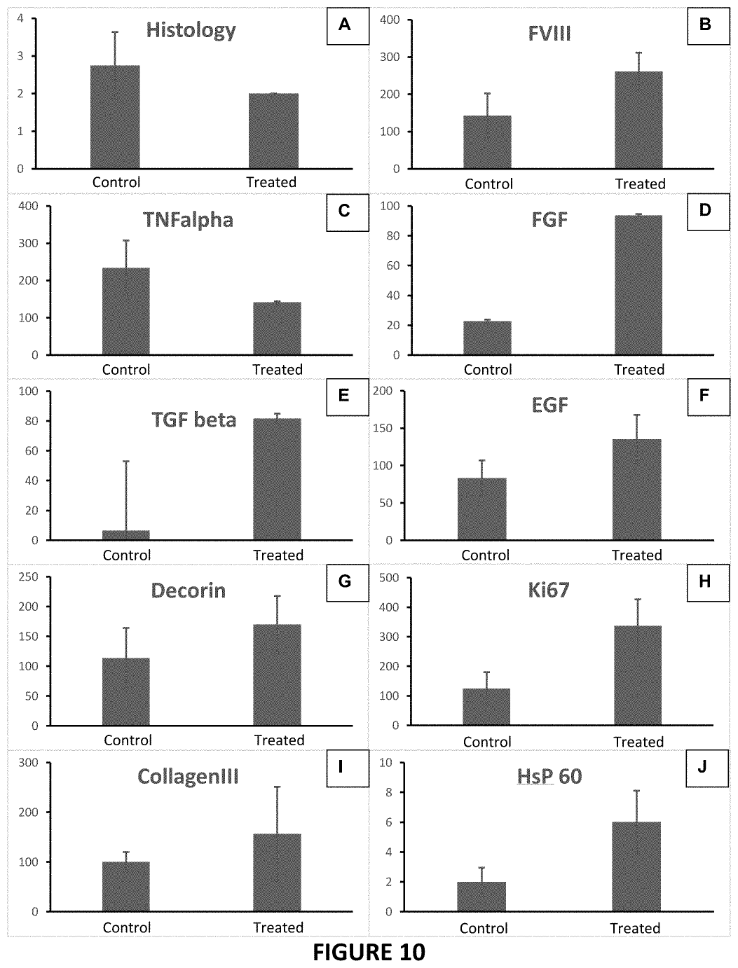

After re-epithelization, the skin treated with the biophotonic treatment showed a heavy concentration of follicles as well as a healthy pilosebaceous apparatus (FIG. 2, panel B). A large number of follicles with an evident papilla containing capillaries were observed in the treated skin (FIG. 2, panel B), in contrast to the non-treated skin which showed a lower number of follicles (FIG. 2, panel A). Cells of the treated skin also demonstrated a higher number of anagenic follicles and a higher VEGF expression than cells of the non-treated skin (FIGS. 3A and 3B). A high power magnification of the follicular area of the dermis of the treated skin after re-epithelization showed follicles characterized by an evident vascular papilla with capillaries (FIG. 4, panel B), whereas catagenic follicles were observed in the non-treated skin (FIG. 4, panel A). EGF was strongly expressed by the cells of the hair bulb of treated skin (FIG. 4, panel B) in contrast to the cells of the bulb's papilla in the non treated skin which showed lower EGF expression (FIG. 4, panel A). EGF was also highly expressed in the epidermis and in the hair follicles of treated skin (FIG. 5, panel A) Immunohistochemistry evaluation of VEGF expression showed a large number of new blood vessels in the dermis of the treated skin indicating hair regrowth (FIG. 5, panel B). In addition, immunohistochemistry evaluation of Ki67 (Mib/1) expression showed a strong and continuous expression of Ki67 in the treated skin indicating an active status of replication of the epidermis and adnexa cells (FIG. 5, panel C). Follicles with vascula pailla and regrowing aspect were also observed in the treated skin (FIG. 5, panel D). The results of immunohistochemistry performed on the skin samples obtained from subjects 1, 2, 3, 4 and 5 are presented in FIGS. 6, 7, 8, 9 and 10. Taken together these data indicate that EGF and Ki67(Mib/1) were initially co-expressed in the developing hair follicle, however expression of EGF was limited to the matrix cells and disappeared during catagen. Expression of Ki67 in the hair follicle was more dynamic; first it was expressed only in the matrix and precortical cells, it was then expressed only in limited area of matrix cells and tended to disappear in late catagen. During anagen, germinative matrix cells proliferated to generate progenitor cells, which received signals from the dermal papilla to differentiate into either hair shaft cells or inner root sheath cells. During catagen, hair production ceased and hair follicle degenerated to form a club hair. During telogen, hair follicle rested and at the end of telogen, dermal papilla at the base of the hair follicle interacted with the adjacent bulge region to initiate the second round of hair follicle morphogenesis. The observed neo-angiogenesis, oxygen increase, and increase of growth factors in the treated skin versus the non treated skin indicate a regrowth of hair and a reduction of some of the adverse factors that are generally associated to hair loss, such as, for example, folliculitis, sebaceous glands inflammation that alters the glands secretion and bacterial/yeast growth.

Overall, these data indicate that the biophotonic compositions and the biophotonic treatments of the present disclosure are capable of enhancing hair growth.

All references cited in this specification, and their references, are incorporated by reference herein in their entirety where appropriate for teachings of additional or alternative details, features, and/or technical background.

While the disclosure has been particularly shown and described with reference to particular embodiments, it will be appreciated that variations of the above-disclosed and other features and functions, or alternatives thereof, may be desirably combined into many other different systems or applications. Also, that various presently unforeseen or unanticipated alternatives, modifications, variations or improvements therein may be subsequently made by those skilled in the art which are also intended to be encompassed by the following claims.

1. A method for enhancing hair growth on an area of a skin of a subject, comprising: topically applying a biophotonic composition comprising at least one light-absorbing molecule to the skin area; and exposing said applied biophotonic composition to actinic light to cause activation of the biophotonic composition, wherein activation of the applied biophotonic composition triggers enhancement of hair growth.

2. (canceled)

3. The method according to claim 1, wherein the biophotonic composition is a biophotonic membrane.

4. The method according to claim 1, wherein the enhancement of hair growth is achieved non-systemically.

5.-6. (canceled)

7. The method according to claim 1, wherein the subject suffers from hair loss.

8. The method according to claim 1, wherein the subject suffers from alopecia.

9. The method according to claim 8, wherein the alopecia is selected from androgenetic alopecia, alopecia areata, anagen effluvium, self-induced hair loss, telogen effluvium, or scarring alopecia.

10. (canceled)

11. The method according to claim 1, wherein the at least one light-absorbing molecule is selected from eosin, erythrosine, and fluorescein.

12. (canceled)

13. The method according to claim 11, wherein the at least one light-absorbing molecule is eosin Y.

14. The method according to claim 1, wherein the at least one light-absorbing molecule is an endogeneous light-absorbing molecule.

15. The method according to claim 1, wherein the at least one light-absorbing molecule is 7,8-Dimethyl-10-[(2S,3S,4R)-2,3,4,5-tetrahydroxypentyl]benzo[g]pteridine-2,4-dione

16. The method according to claim 1, wherein the at least one light-absorbing molecule is present in an amount of at least about 0.02% by weight of the composition.

17.-22. (canceled)

23. The method according to claim 1, wherein the composition further comprises at least one hydrophilic gelling agent.

24. The method according to claim 23, wherein the hydrophilic gelling agent is selected from glucose, modified starch, methyl cellulose, carboxymethyl cellulose, propyl cellulose, hydroxypropyl cellulose, Carbopol® polymers, alginic acid, sodium alginate, potassium alginate, ammonium alginate, calcium alginate, agar, carrageenan, locust bean gum, pectin, and gelatin.

25. The method according to claim 1, wherein the composition is exposed to actinic light for at least one illumination period of from about 1 minute to about 9 minutes per cm² of an area to be treated.

26. The method according to claim 1, wherein said composition is exposed to actinic light for at least one illumination period of from about 1 second to about 60 seconds per cm² of an area to be treated.

27. The method according to claim 1, wherein said composition is exposed to actinic light for at least one illumination period of about 5 minutes per cm² of an area to be treated.

28. The method according to claim 1, wherein said composition is exposed to actinic light for at least two illumination periods.

29. The method according to claim 28, wherein said composition is exposed to actinic light for at least two treatment periods, and wherein each period is followed by a resting interval.

30. (canceled)

31. The method according to claim 1, further comprising: topically applying the composition to the skin in need of hair growth enhancement; exposing the skin to actinic light for an illumination period of from about 1 minute to about 10 minutes; removing the source of actinic light away from the treated skin for a resting interval of from about 1 minute to about 5 minutes; and exposing the treated skin to actinic light for a second illumination period of from about 1 minute to about 10 minutes; and wherein the first exposure to actinic light activates the composition.

32.-38. (canceled)

39. A method for inducing hair follicles of a skin area into anagen, comprising: topically applying a composition comprising at least one light-absorbing molecule to the skin area; and exposing said applied composition to actinic light to cause activation of the composition, wherein activation of the applied composition induces hair follicles of the skin area into anagen.

40.-42. (canceled)
